(19) United States
(12) Patent Application Publication
(10) Pub. No.: US 2024/0350660 A1
(43) Pub. Date: Oct. 24, 2024

(54) Compositions and Methods for Increasing Bioavailability Using Cyclodextrin Modification

(71) Applicant: HAUS BIOCEUTICALS INC., Oklahoma City, OK (US)

(72) Inventors: Michael Centola, Oklahoma City, OK (US); Philip ALEX, Oklahoma City, OK (US)

(21) Appl. No.: 18/684,291

(22) PCT Filed: Aug. 19, 2022
(86) PCT No.: PCT/US2022/075218

§ 371 Date: Feb. 16, 2024

Related U.S. Application Data
(60) Provisional application No. 63/234,806, filed on Aug. 19, 2021.

(51) Int. Cl. A61K 47/69; A61P 3/10; A61P 17/00; A61P 31/14

(52) U.S. Cl. CPC A61K 47/6951; A61P 3/10; A61P 17/00; A61P 31/14

(57) ABSTRACT

Compositions and methods are disclosed for enhancing the bioavailability and/or increasing the stability of at least one agent by modifying the at least one agent with cyclodextrin. Pharmaceutical compositions including the cyclodextrin-modified agents are disclosed. Methods of manufacturing the cyclodextrin-modified agents as well as methods of using the cyclodextrin-modified agents are also disclosed.

### CROSS REFERENCE TO RELATED APPLICATIONS/INCORPORATION BY REFERENCE

The subject application claims benefit under 35 USC § 119 (e) of U.S. Ser. No. 63/234,806, filed Aug. 19, 2021. The entire contents of the above-referenced patent application(s) are hereby expressly incorporated herein by reference.

### STATEMENT REGARDING FEDERALLY SPONSORED RESEARCH OR DEVELOPMENT

### Not Applicable.

### BACKGROUND

Many active agents with desired biological activities exhibit undesirable levels of bioavailability and stability when administered to a patient. Thus, different carrier materials are continually being developed to overcome the undesirable properties of drug molecules.

Various methods of increasing bioavailability/stability are currently known in the art. These methods include binding/complexation with a cyclodextrin, lipid-based encapsulation (such as, but not limited to, emulsion/nanoemulsion, solid lipid nanoparticles, liposomes/nanoliposomes, etc.), phospholipid encapsulation, protein and amino acid encapsulation, binding/complexation to nanoparticles, use of adjuvants (such as, but not limited to, piperine and black pepper), and the like.

One currently used method to increase bioavailability includes the use of a cyclodextrin. Cyclodextrins are a family of cyclic oligosaccharides that possess a hydrophilic outer surface and a lipophilic central cavity or pocket. In the present method, a substance becomes bound, entrapped, and/or complexed within the central pocket of the cyclodextrin, and such binding increases the water solubility and stability of the substance.

However, for most active agents, current methods of manufacturing cyclodextrin complexes were not optimized for a given agent. Instead, established, generic methods have been employed, resulting in limited clinical potential. Therefore, there is a need in the art for new and improved methods that include optimization of the variables associated with manufacturing cyclodextrin complexes, thereby resulting in significantly improved clinical activity relative to established methods. It is to such methods, and compositions utilized therein and/or formed therefrom, that the present disclosure is directed. In particular, the methods of the present disclosure optimize the methods for manufacturing cyclodextrin complexes for a given agent by employing a clinically relevant biological assay (e.g., a preclinical model of human disease) to quantitate the impact of varying manufacturing parameters on the clinical activity of the cyclodextrin complex.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function.

FIG. 1 graphically depicts a comparison of the body weight observed in a human ACE2 transgenic COVID-19 mouse model utilizing native anti-PHR agents and high bioavailability anti-PHR agents produced in accordance with the present disclosure. All treatments were administered daily, starting at Day 2 post infection.

FIG. 2 graphically depicts a comparison of survival rate observed in the COVID-19 mouse model utilizing native anti-PHR agents and high bioavailability anti-PHR agents produced in accordance with the present disclosure. All treatments were administered daily, starting at Day 2 post infection.

FIG. 3 graphically depicts the ability of the anti-PHR agent rutin and cyclodextrin-modified forms thereof to inhibit SARS-COV-2 replication in vitro. “UNRX”—Untreated. “1:1 BCDRUT”—Betacyclodextrin Rutin, produced with a 1:1 molar ratio of betacyclodextrin to rutin; cells treated at 1 μM final concentration. “1:1 HPGCDRUT”—Hydroxypropylgammacyclodextrin Rutin produced with a 1:1 molar ratio of hydroxypropylgammacyclodextrin to rutin; cells treated at 1 μM final concentration. “1:1 GCDRUT”-Gammacyclodextrin Rutin produced with a 1:1 molar ratio of gammacyclodextrin to rutin; cells treated at 1 μM final concentration. “1:1 ACDRUT”—Alphacyclodextrin Rutin produced with a 1:1 molar ratio of alphacyclodextrin to rutin; cells treated at 1 μM final concentration. “RUT”—Rutin, no cyclodextrin; cells treated at 1 μM final concentration.

FIG. 4 graphically depicts the ability of the anti-PHR agent rutin and cyclodextrin-modified forms thereof to inhibit SARS-COV-2 replication in vitro. “2:1 BCDRUT”—Betacyclodextrin Rutin produced with a 2:1 molar ratio of betacyclodetrin to rutin. “1:1 BCDRUT”—Betacyclodextrin Rutin produced with a 1:1 molar ratio of betacyclodetrin to rutin. “2:1 BCDRUT”—Hydroxypropylbetacyclodextrin Rutin produced with a 2:1 molar ratio of Hydroxypropylbetacyclodetrin to rutin. “1:1 BCDRUT”—Hydroxypropylbetacyclodextrin Rutin produced with a 1:1 molar ratio of Hydroxypropylbetacyclodetrin to rutin. “RUT”—Rutin, no cyclodextrin; 1, 10, and 100 correspond to 1, 10, and 100 mg/ml treatment concentrations, respectively.

FIG. 5 graphically depicts a disease activity score for two non-limiting embodiments of cyclodextrin-modified anti-PHR agents constructed in accordance with the present disclosure (cyclodextrin-modified tetrahydrocurcumin (BCD-THC) and cyclodextrin-modified rutin (BCDRUT) compared to untreated (Unrx) and unmodified tetrahydrocurcumin (THC) in a murine model of chronic atopic dermatitis.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, kits, assemblies, and/or methods disclosed herein can be made and executed without undue experimentation in light of the present disclosure. While the compositions, kits, assemblies, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically acceptable excipient” refers to any carrier, vehicle, and/or diluent known in the art or otherwise contemplated herein that may improve solubility, deliverability, dispersion, stability, and/or conformational integrity of the compositions disclosed herein.

The terms “patient” and “subject” are used herein interchangeably and will be understood to include human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes. The term “treatment” includes a detectable or measurable improvement in a subject's condition and/or at least one symptom thereof.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one infection or condition. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

In addition, an “effective amount” of an active agent of the present disclosure refers to an amount which is effective in controlling, reducing, or inhibiting a condition as described herein, such as (but not limited to) a viral infection and/or the effects associated therewith. The term “controlling” is intended to refer to all processes wherein there may be a slowing, interrupting, arresting, or stopping of the progression of the condition and does not necessarily indicate a total elimination of the symptoms of the condition.

The term “effective amount” is further meant to define an amount resulting in the improvement of any parameters or clinical symptoms characteristic of a condition. The actual dose will vary with the patient's overall condition, the seriousness of the condition or symptoms, and counter indications. As used herein, the term “effective amount” also means the total amount of each active agent (component) of the pharmaceutical composition or method that is sufficient to show a meaningful patient benefit, i.e., reduction of a condition. When applied to an individual active ingredient, administered alone, the term refers to that ingredient alone. When applied to a combination, the term refers to combined amounts of the active agent(s) that results in the therapeutic effect, whether administered in combination, serially or simultaneously.

The term “ameliorate” means a detectable or measurable improvement in a subject's condition or symptom thereof. A detectable or measurable improvement includes a subjective or objective decrease, reduction, inhibition, suppression, limit, or control in the occurrence, frequency, severity, progression, or duration of the condition, or an improvement in a symptom or an underlying cause or a consequence of the condition, or a reversal of the condition. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most, or all adverse symptoms, complications, consequences, or underlying causes associated with the condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition (e.g., stabilizing), over a short or long duration of time (e.g., seconds, minutes, hours).

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concepts, compositions and methods are provided for the binding/complexation of an agent with a cyclodextrin to enhance the bioavailability and/or stability thereof.

Certain non-limiting embodiments of the present disclosure are directed to a method of producing a cyclodextrin (CD)-modified agent. The method includes the steps of: combining at least one of any of the agents disclosed or otherwise contemplated herein with a cyclodextrin to form a mixture; incubating the mixture at a temperature above room temperature and at a pressure above atmospheric pressure for a sufficient amount of time to form a CD-modified agent. The CD-modified agent produced in this manner has enhanced bioavailability when compared to the agent alone.

Any type of agent that could benefit from increased bioavailability and that is capable of being bound/complexed with cyclodextrin can be utilized in accordance with the present disclosure. Non-limiting types of agents that may be utilized in accordance with the present disclosure include anti-PHR agents, guaiazulene, zinc, catechin, licorice extract, glycyrrhetinic acid, clofazimine, quinine, niclosamide, amodiquine diydrochloride, and the like, as well as any combinations thereof.

In particular (but non-limiting) embodiments, the agent is an anti-PHR (anti-Pathogenic Host Response) agent selected from the group consisting of rutin, sodium rutin, curcumin, tetrahydrocurcumin, quercetin, hesperidin, baicalin, green tea extract, rose extract, betulinic acid, tannic acid, bisabolol, a modified form thereof, and combinations thereof (including, but not limited to, at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, or more of the above agents and/or modified forms thereof).

Non-limiting examples of cyclodextrins that may be utilized in accordance with the present disclosure include alpha-cyclodextrin (ACD), beta-cyclodextrin (BCD), hydroxyethyl beta-cyclodextrin (HEBCD), hydroxypropyl beta-cyclodextrin (HPBCD), sulfobutyl beta-cyclodextrin (SBCD), gamma-cyclodextrin (GCD), hydroxypropyl gamma-cyclodextrin (HPGD), and the like.

The mixture may be incubated at any temperature above room temperature (i.e., above about 25° C.) that allows for the production of a CD-modified agent with enhanced bioavailability. Non-limiting temperatures that may be utilized include about 20° C., about 25° C., about 30° C., about 35° C., about 40° C., about 45° C., about 50° C., about 55° C., about 60° C., about 65° C., about 70° C., about 75° C., about 80° C., about 90° C., about 95° C., about 100° C., about 110°° C., about 120°° C., about 130° C., about 140° C., about 150°° C., about 160°° C., about 170° C., about 180°° C., about 190° C., about 200°° C., about 210°° C., about 220° C., about 230° C., about 240° C., about 250° C., about 260° C., about 270° C., about 280° C., about 290° C., about 300°° C., about 310° C., about 320° C., about 330° C., about 340° C., about 350° C., about 360°° C., about 370° C., about 380° C., about 390°° C., about 400° C., about 410° C., about 420° C., about 430° C., about 440° C., about 450° C., about 460° C., about 470° C., about 480°° C., about 490° C., about 500° C., or higher, as well as a range formed of two of the above values (i.e., a range of from about 30° C. to about 500° C., a range of from about 40° C. about to about 300° C., etc.), or a range formed of two values that each fall within two of the above values (i.e., a range of from about 37° C. to about 295° C., etc.).

The mixture may be incubated at any pressure above atmospheric pressure (i.e., above about 1 PSI) that allows for the production of a CD-modified agent with enhanced bioavailability. Non-limiting pressures that may be utilized include about 2 PSI, about 3 PSI, about 4 PSI, about 5 PSI, about 6 PSI, about 7 PSI, about 8 PSI, about 9 PSI, about 10 PSI, about 11 PSI, about 12 PSI, about 13 PSI, about 14 PSI, about 15 PSI, about 16 PSI, about 17 PSI, about 18 PSI, about 19 PSI, about 20 PSI, about 25 PSI, about 30 PSI, about 35 PSI, about 40 PSI, about 45 PSI, about 50 PSI, about 55 PSI, about 60 PSI, about 65 PSI, about 70 PSI, about 75 PSI, about 80 PSI, about 85 PSI, about 90 PSI, about 95 PSI, about 100 PSI, about 110 PSI, about 120 PSI, about 130 PSI, about 140 PSI, about 150 PSI, about 160 PSI, about 170 PSI, about 180 PSI, about 190 PSI, about 200 PSI, about 210 PSI, about 220 PSI, about 230 PSI, about 240 PSI, about 250 PSI, about 260 PSI, about 270 PSI, about 280 PSI, about 290 PSI, about 300 PSI, about 350 PSI, about 400 PSI, about 450 PSI, about 500 PSI, or higher, as well as a range formed of two of the above values (i.e., a range of from about 2 PSI to about 300 PSI, a range of from about 15 PSI to about 200 PSI, etc.), or a range formed of two values that each fall within two of the above values (i.e., a range of from about 9.5 PSI to about 325 PSI, etc.).

The mixture may be incubated for any time period that allows for the production of a CD-modified agent with enhanced bioavailability. Non-limiting amounts of time that may be utilized include about 1 second, about 30 seconds, about 1 minute, about 2 minutes, about 3 minutes, about 4 minutes, about 5 minutes, about 6 minutes (i.e., about 0.1 hour), about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 1.5 hours, about 2 hours, about 2.5 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, or longer, as well as a range formed of two of the above values (i.e., a range of from about 1 minute to about 24 hours, a range of from about 0.1 hour to about 18 hours, a range of from about 0.1 hour to about 1 hour, etc.), or a range formed of two values that each fall within two of the above values (i.e., a range of from about 1.5 hours to about 20.5 hours, a range of from about 12 minutes to about 3.5 hours, etc.).

The cyclodextrin and agent may be combined to form the mixture at any concentrations and at any molar ratio that allow for the production of a CD-modified agent with enhanced bioavailability in accordance with the present disclosure. Non-limiting amounts of cyclodextrin: agent molar ratios that may be utilized include about 0.1:1, about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, about 4:1, about 4.1:1, about 4.2:1, about 4.3:1, about 4.4:1, about 4.5:1, about 4.6:1, about 4.7:1, about 4.8:1, about 4.9:1, about 5:1, or higher, as well as a range formed of two of the above values (i.e., a range of from about 1:1 to about 3:1, etc.), or a range formed of two values that each fall within two of the above values (i.e., a range of from about 0.75:1 to about 2.25:1, etc.).

Non-limiting concentrations of cyclodextrin or agent that may be utilized include about 0.0001 wt %, about 0.005 wt %, about 0.001 wt %, about 0.005 wt %, about 0.01 wt %, about 0.05 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, and about 2 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 13 wt %, about 14 wt %, about 15 wt %, about 16 wt %, about 17wt %, about 18 wt %, about 19 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or higher, as well as a concentration that falls within a range formed of two of the above values (i.e., a range of from about 0.001 wt % to about 75 wt %, a range of from about 0.05 wt % to about 35 wt %, etc.), or a concentration that falls within a range of two values, each of which falls between two values listed above (i.e., a range of from about 0.03 wt % to about 22 wt %; a range of from about 0.08 wt % to about 63 wt %; etc.).

In addition, non-limiting examples of molar concentrations of cyclodextrin and agent that may be utilized in accordance with the present disclosure include about 0.0001 M, about 0.0005 M, about 0.001 M, about 0.005 M, about 0.01 M, about 0.05 M, about 0.1 M, about 0.2 M, about 0.3 M, about 0.4 M, about 0.5 M, about 0.6 M, about 0.7 M, about 0.8 M, about 0.9 M, about 1 M, about 2 M, about 3 M, about 4 M, about 5 M, or higher, or a molar concentration that falls within a range formed of two of the above values (i.e., a range of from about 0.0001 M to about 1 M, a range of from about 0.001 M to about 0.1 M, etc.), or a concentration that falls within a range of two values, each of which falls between two values listed above (i.e., a range of from about 0.007 M to about 0.86 M, etc.).

In certain particular (but non-limiting) embodiments, the agent and/or the cyclodextrin may be combined with a solvent prior to or at the same time as the agent and cyclodextrin are combined with one another to form the mixture. Any solvents known in the art or otherwise contemplated herein that allow for the production of a CD-modified agent with enhanced bioavailability in accordance with the present disclosure. Non-limiting examples of solvents that may be utilized include ethanol, water, methanol, acetone, and combinations thereof.

Certain non-limiting embodiments of the present disclosure are directed to compositions comprising cyclodextrin-modified agents. Any type of agent that could benefit from increased bioavailability and that is capable of being bound/complexed with cyclodextrin can be utilized in accordance with the present disclosure. Non-limiting types of agents that may be utilized in accordance with the present disclosure include anti-PHR agents (such as, but not limited to, rutin, sodium rutin, curcumin, tetrahydrocurcumin, quercetin, hesperidin, baicalin, green tea extract, rose extract, betulinic acid, tannic acid, bisabolol, a modified form thereof, and combinations thereof), guaiazulene, zinc, catechin, licorice extract, glycyrrhetinic acid, clofazimine, quinine, niclosamide, amodiquine diydrochloride, and the like, as well as any combinations thereof. Non-limiting examples of cyclodextrins that may be utilized in accordance with the present disclosure include alpha-cyclodextrin (ACD), beta-cyclodextrin (BCD), hydroxyethyl beta-cyclodextrin (HEBCD), hydroxypropyl beta-cyclodextrin (HPBCD), sulfobutyl beta-cyclodextrin (SBCD), gamma-cyclodextrin (GCD), hydroxypropyl gamma-cyclodextrin (HPGD), and the like.

Non-limiting examples of cyclodextrin-modified agents include ACD-curcumin, BCD-curcumin, HEBCD-curcumin, HPBCD-curcumin, SBCD-curcumin, GCD-curcumin, HPGD-curcumin, ACD-quercetin, BCD-quercetin, HEBCD-quercetin, HPBCD-quercetin, SBCD-quercetin, GCD-quercetin, HPGD-quercetin, ACD-rutin, BCD-rutin, HEBCD-rutin, HPBCD-rutin, SBCD-rutin, GCD-rutin, HPGD-rutin, and the like, as well as any combinations thereof.

The cyclodextrin-modified agents of the present disclosure may be produced by any of the methods disclosed or otherwise contemplated herein. However, it will be understood that the cyclodextrin-modified agents are not limited to production by these novel methods; rather, the scope of the present disclosure also includes production of these cyclodextrin-modified agents by any known or otherwise contemplated methods of cyclodextrin modification.

Certain non-limiting embodiments of the present disclosure are directed to pharmaceutical compositions that comprise one or more of any of the cyclodextrin-modified agents produced in accordance with the present disclosure (either alone or in combination with one or more additional agents) combined with a pharmaceutically acceptable carrier. For example (but not by way of limitation), the pharmaceutical composition may contain, in addition to the cyclodextrin-modified agent(s), one or more of a diluent, an excipient, a filler, a salt, a buffer, a stabilizer, a solubilizer, a vehicle, and other materials well known in the art, as well as any combination thereof. Suitable carriers, vehicles, and other components for pharmaceutical formulations are described, for example, in Remington: The Science and Practice of Pharmacy, 23rd ed (2020).

Where used herein, the term “pharmaceutically acceptable carrier” refers to any compound used in combination (e.g., in a composition or formulation) with the cyclodextrin-modified agent(s) of the present disclosure, for example, for aiding in delivery of the agent(s) to the subject to be treated. The term “pharmaceutically acceptable” means a non-toxic material that does not interfere with the effectiveness of the biological activity of the active agent(s). The characteristics of the carrier will depend on the route of administration.

For example, but not by way of limitation, the agent(s) may be dissolved (separately or together) in a physiologically acceptable pharmaceutical carrier and administered as either a solution or a suspension. Non-limiting examples of suitable pharmaceutically acceptable carriers include water; saline; dextrose solutions; fructose solutions; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; or any combination thereof. A sterile diluent, which may contain materials generally recognized for approximating physiological conditions and/or as required by governmental regulations, may be employed as the pharmaceutically acceptable carrier. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use.

The pharmaceutical compositions may also contain one or more additional components in addition to the cyclodextrin-modified agent(s) (and pharmaceutically acceptable carrier(s), if present). Examples of additional secondary compounds that may be present include, but are not limited to, diluents, fillers, salts, buffers, preservatives, stabilizers, solubilizers, wetting agents, emulsifying agents, dispersing agents, and other materials well known in the art.

The cyclodextrin-modified agent(s) may be present in the same or different pharmaceutical compositions at any concentration that allows the pharmaceutical composition(s) to function in accordance with the present disclosure; for example, but not by way of limitation, the cyclodextrin-modified agent(s) may each be present at a concentration of about 0.0001 wt %, about 0.005 wt %, about 0.001 wt %, about 0.005 wt %, about 0.01 wt %, about 0.05 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, and about 2 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 11 wt %, about 12 wt %, about 13 wt %, about 14 wt %, about 15 wt %, about 16 wt %, about 17wt %, about 18 wt %, about 19 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or higher. In addition, the cyclodextrin-modified agent(s) may each be present at a concentration that falls within a range formed of two of the above values (i.e., a range of from about 0.001 wt % to about 75 wt %, a range of from about 0.05 wt % to about 35 wt %, etc.); also, the cyclodextrin-modified agent(s) may be present at a concentration that falls within a range of two values, each of which falls between two values listed above (i.e., a range of from about 0.03 wt % to about 22 wt %; a range of from about 0.08 wt % to about 63 wt %; etc.).

In addition, in certain non-limiting embodiments, the cyclodextrin-modified agent(s) may each be present in the pharmaceutical compositions at a specific molar concentration. Non-limiting examples of molar concentrations that may be utilized in accordance with the present disclosure include about 0.0001 M, about 0.0005 M, about 0.001 M, about 0.005 M, about 0.01 M, about 0.05 M, about 0.1 M, about 0.2 M, about 0.3 M, about 0.4 M, about 0.5 M, about 0.6 M, about 0.7 M, about 0.8 M, about 0.9 M, about 1 M, about 2 M, about 3 M, about 4 M, about 5 M, or higher. In addition, the cyclodextrin-modified agent(s) may each be present at a molar concentration that falls within a range formed of two of the above values (i.e., a range of from about 0.0001 M to about 1 M, a range of from about 0.001 M to about 0.1 M, etc.); also, the cyclodextrin-modified agent(s) may each be present at a concentration that falls within a range of two values, each of which falls between two values listed above (i.e., a range of from about 0.007 M to about 0.86 M, etc.).

The amount of each of the cyclodextrin-modified agent(s) present in the pharmaceutical composition(s) that is effective in the treatment described herein can be determined by the attending diagnostician, as one of ordinary skill in the art, by the use of conventional techniques and by observing results obtained under analogous circumstances. determining the therapeutically effective dose, a number of factors may be considered by the attending diagnostician, including, but not limited to: the species of the subject; its size, age, and general health; the specific diseases, infections, and/or other conditions involved; the degree, involvement, and/or severity of the diseases, infections, and/or conditions; the response of the individual subject; the particular cyclodextrin-modified agent(s) administered; the mode of administration; the dose regimen selected; the use of concomitant medication; and other relevant circumstances. A therapeutically effective amount of each of the cyclodextrin-modified agent(s) of the present disclosure also refers to an amount of each of the cyclodextrin-modified agent(s) which is effective in controlling, reducing, or ameliorating the condition/infection/disorder/disease to be treated or may refer to the amount of the cyclodextrin-modified agent(s) required to achieve a prophylactic effect for the purpose of preventing, controlling, reducing, or ameliorating the condition/infection/disorder/disease to be treated.

For example, but not by way of limitation, the therapeutically effective amount of pharmaceutical composition(s) will generally contain sufficient cyclodextrin-modified agent(s) to deliver each agent in a range of from about 0.01 μg/kg to about 10 g/kg (weight of cyclodextrin-modified agent(s)/body weight of patient). For example, but not by way of limitation, the composition will deliver about 0.1 μg/kg to about 1 g/kg, and more particularly about 1 μg/kg to about 500 mg/kg of the cyclodextrin-modified agent(s).

Exemplary, non-limiting therapeutically or prophylactically effective amounts of each of the cyclodextrin-modified agent(s), based on the subject's body weight, include about 0.01 μg/kg, about 0.05 μg/kg, about 0.1 μg/kg, about 0.5 μg/kg, about 1 μg/kg, about 5 μg/kg, about 10 μg/kg, about 50 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 75 mg/kg, about 80 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1 g/kg, and higher, as well as a range formed from two of the above values (i.e., a range of from about 1 μg/kg to about 100 mg/kg of the subject's body weight, a range of from about 1 μg/kg to about 500 mg/kg of the subject's body weight, etc.), as well as a range formed from two values, each of which falls between two of the above values (i.e., a range of from about 7 μg/kg to about 575 mg/kg, a range of from about 2 μg/kg to about 325 mg/kg, etc.).

Each pharmaceutical composition is formulated to contain an effective amount of the cyclodextrin-modified agent(s), wherein the amount depends on the animal to be treated and the condition to be treated. In certain embodiments, each of the cyclodextrin-modified agent(s) is administered at a dose ranging from about 0.001 mg to about 100 g, a dose ranging from about 0.01 mg to about 10 g, a dose ranging from about 0.1 mg to about 10 g, a dose ranging from about 1 mg to about 10 g, a dose ranging from about 1 mg to about 9 g, a dose ranging from about 1 mg to about 8 g, a dose ranging from about 1 mg to about 7 g, a dose ranging from about 1 mg to about 6 g, a dose ranging from about 1 mg to about 5 g, a dose ranging from about 10 mg to about 10 g, a dose ranging from about 50 mg to about 5 g, a dose ranging from about 50 mg to about 5 g, a dose ranging from about 50 mg to about 2 g, a dose ranging from about 0.05 μg to about 1.5 mg, a dose ranging from about 10 μg to about 1 mg, a dose ranging from about 30 μg to about 500 μg, a dose ranging from about 0.1 μg to about 200 mg, a dose ranging from about 0.1 μg to about 5 μg, a dose ranging from about 5 μg to about 10 μg, a dose ranging from about 10 μg to about 25 μg, a dose ranging from about 25 μg to about 50 μg, a dose ranging from about 50 μg to about 100 μg, a dose ranging from about 100 μg to about 500 μg, a dose ranging from about 500 μg to about 1 mg, or a dose ranging from about 1 mg to about 2 mg. The specific dosage level for any particular subject depends upon a variety of factors including the activity of the specific peptide, the age, body weight, general health, sex, diet, time of administration, route of administration, and rate of excretion, drug combination, and the severity of the particular disease undergoing therapy.

In certain particular (but non-limiting) embodiments of the present disclosure, the dose of each cyclodextrin-modified agent is in a range of from about 1 μg/kg to about 500 mg/kg.

The dosage of an administered pharmaceutical composition for the subject will vary depending upon such factors as the patient's age, weight, height, sex, general medical condition, and previous medical history. In certain non-limiting embodiments, the recipient is provided with a dosage of each of the cyclodextrin-modified agent(s) that is in the range of from about 1 mg to about 1000 mg as a single infusion or single or multiple injections, although a lower or higher dosage also may be administered. The dosage may be in the range of from about 25 mg to about 100 mg of the cyclodextrin-modified agent(s) per square meter (m²) of body surface area for a typical adult, although a lower or higher dosage also may be administered. Examples of dosages that may be administered to a human subject further include, for example, in a range of from about 1 mg to about 500 mg, a range of from about 1 mg to about 70 mg, or a range of from about 1 mg to about 20 mg, although higher or lower doses may be used.

Dosages may be repeated as needed, for example (but not by way of limitation), once every 10 minutes, once every 30 minutes, once every hour, once every two hours, once every three hours, once every four hours, once every five hours once every six hours, once every eight hours, once every 12 hours, once a day, once per week, etc. It may also be given less frequently, such as once every month, every other week for several months, or more frequently, such as twice weekly, or by continuous infusion.

The cyclodextrin-modified compositions of the present disclosure may be administered via one or more routes of administration using one or more of a variety of methods known in the art. As will be appreciated by the skilled artisan, the route and/or mode of administration will vary depending upon the desired results. Selected routes of administration include (but are not limited to) intravenous, intramuscular, intradermal, intraperitoneal, subcutaneous, spinal, or other parenteral routes of administration, for example by injection or infusion. The cyclodextrin-modified agent(s) can be delivered alone or as pharmaceutical compositions by any means known in the art, e.g., systemically, regionally, or locally; by intra-arterial, intrathecal (IT), intravenous (IV), parenteral, intra-pleural cavity, or local administration, as subcutaneous, intra-tracheal (e.g., by aerosol), or transmucosal (e.g., buccal, bladder, vaginal, uterine, rectal, nasal mucosa). Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. Alternatively, compositions can be administered via a non-parenteral route, such as a topical, epidermal, or mucosal route of administration, for example, intranasally, orally, vaginally, rectally, sublingually, or topically. In one embodiment, the composition(s) is administered by infusion. In another embodiment, the composition(s) is administered subcutaneously. In another embodiment, the composition(s) is administered orally. In another embodiment, the composition(s) is administered to the ear canal. In another embodiment, the composition(s) is administered transdermally. In another embodiment, the composition(s) is administered to the lungs with no penetration, partial penetration, or complete penetration of the lung tissues.

Compositions for inhalation or insufflation include solutions and suspensions in pharmaceutically acceptable, aqueous, or organic solvents, or mixtures thereof, and powders. The liquid or solid compositions may contain suitable pharmaceutically acceptable excipients as described supra.

The compositions can be administered by the oral or nasal respiratory route for local or systemic effect. Compositions in pharmaceutically acceptable solvents may be nebulized by use of inert gases. Nebulized solutions may be inhaled directly from the nebulizing device, or the nebulizing device may be attached to a face mask tent or intermittent positive pressure breathing machine. Solution, suspension, or powder compositions may also be administered orally or nasally from devices which deliver the formulation in an appropriate manner.

When the pharmaceutical composition is to be used as an injectable material, it can be formulated into a conventional injectable carrier. Non-limiting examples of suitable carriers include biocompatible and pharmaceutically acceptable phosphate buffered saline solutions, which are particularly isotonic.

In certain non-limiting embodiments, the cyclodextrin-modified agent(s) is provided as a lyophilized product that is reconstituted, such as (but not limited to) for injection. For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as (but not limited to) sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

Practice of the methods of the present disclosure may include administering to a subject a therapeutically effective amount of the pharmaceutical composition(s) (containing the cyclodextrin-modified agent(s) in any suitable systemic and/or local formulation), in an amount effective to deliver the desired dosage. The dosage can be administered, for example, but not by way of limitation, continuously or intermittently. In addition, the dosage can be administered on a one-time basis or administered at multiple times (for example, but not by way of limitation, from one to five times per day, or once or twice per week). The pharmaceutical composition may be administered either alone or in combination with other therapies, in accordance with the inventive concepts disclosed herein.

Each of the pharmaceutical compositions of the present disclosure can be administered in a single dose treatment or in multiple dose treatments on a schedule and over a time period appropriate to the age, weight, and condition of the subject, the particular composition used, and the route of administration. In one embodiment, a single dose of the composition according to the disclosure is administered. In other embodiments, multiple doses are administered. The frequency of administration can vary depending on any of a variety of factors, e.g., severity of the symptoms, whether the composition is used for prophylactic or curative purposes, etc. For example, in certain non-limiting embodiments, the composition is administered once per day, twice per day, three times per day, four times per day, five times per day, six times per day, seven times per day, eight times per day, nine times per day, 10 times per day, 12 times per day, or more frequently, or the composition may be administered every other day, once per week, twice per week, three times per week, four times per week, five times per week, six times per week, or seven times per week. The duration of treatment, e.g., the period of time over which the composition is administered, can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, or more.

The pharmaceutical compositions of the present disclosure may be administered to a patient to bring about any desired outcome described or otherwise contemplated herein. The pharmaceutical compositions may be utilized to treat or reduce the occurrence of any conditions, diseases, and/or disorders disclosed or otherwise contemplated herein and/or for which the non-modified agents are known to possess activity (ies). That is, if an agent in native/non-modified form is known to possess an anti-disease specific activity, then the pharmaceutical compositions containing cyclodextrin-modified agent will also possess the anti-disease specific activity and can be utilized in a method of treating or reducing the occurrence of said specific disease.

In certain particular (but non-limiting) embodiments, the pharmaceutical compositions may be administered in a method of treating or reducing the occurrence of diabetes in a patient.

In certain particular (but non-limiting) embodiments, the pharmaceutical compositions may be administered in a method of treating or reducing the occurrence of a skin condition in a patient.

In certain particular (but non-limiting) embodiments, the pharmaceutical compositions may be administered in a method of treating or reducing the occurrence of a viral infection in a patient. The methods of the present disclosure may be utilized to treat or reduce the occurrence of viral infections caused by any viruses known in the art or otherwise contemplated herein. Non-limiting examples of viruses that can be treated in accordance with the present disclosure include adenoviruses, astroviruses, coronaviruses (such as, but not limited to, severe acute respiratory syndrome coronavirus (SARS-COV) or Middle East respiratory syndrome coronavirus (MERS-COV)), Coxsackie viruses, cytomegaloviruses (CMV), Ebola viruses, echoviruses, encephalitis viruses, enteroviruses, Epstein-Barr viruses (EBV), erythroviruses, hantaviruses, hepatitis is viruses, herpes viruses (HSV), human immunodeficiency viruses (HIV), influenza viruses, noroviruses, papilloma viruses, parainfluenza viruses, paramyxoviruses, polio viruses, rabies viruses, respiratory syncytial viruses (RSV), rhinoviruses, rotoviruses, rubella viruses, rubeola viruses, Swine flu (H1N1) viruses, Varicella-Zoster viruses, West Nile viruses, Zika viruses, hemorrhagic fever viruses, yellow fever viruses, dengue viruses, and the like, as well as variants thereof.

For example, but not by way of limitation, the compositions and methods may be utilized to treat a coronavirus infection. Non-limiting examples of coronaviruses that cause infections that can be treated in accordance with the present disclosure include severe acute respiratory syndrome coronavirus (SARS-COV), severe acute respiratory syndrome coronavirus 2 (SARS-COV-2, the virus that causes COVID-19), Middle East respiratory syndrome coronavirus (MERS-COV), human coronavirus 229E (HCOV-229E), human coronavirus OC43 (HCoV-OC43), human coronavirus NL63 (HCOV-NL63), and human coronavirus HKU1 (HCoV-HKU1), as well as variants thereof.

In a particular (but non-limiting) embodiment, the compositions and methods of the present disclosure are utilized to treat infection with SARS-COV-2 or a variant thereof.

In certain embodiments, the compositions and methods may be utilized to treat a secondary bacterial infection. Primary viral infections have been shown to lead to increased susceptibility to secondary bacterial infections. For example, secondary bacterial pneumonia is the most frequent complication of the influenza virus infection.

In certain other particular (but non-limiting) embodiments, the pharmaceutical compositions may be administered in a method of treating or reducing the occurrence of one or more conditions/diseases/disorders selected from the group consisting of eczema, scabies, scleroderma, psoriasis, vitiligo, radiation dermatitis, Alzheimer's disease, Parkinson's disease, Dejerine-Sottas disease, cognitive decline, headache, multiple sclerosis, schizophrenia, epilepsy, migraine, depression, major depressive disorder, anxiety, atherosclerosis, hypolipidemia, myocardial infarction, Crohn's disease, ulcerative colitis, irritable bowel disease, pancreatitis, gastric ulcer, duodenal ulcer, hepatitis, arthritis, rheumatoid arthritis, osteoarthritis, lupus, arthralgia, fibromyalgia, psoriatic arthritis, osteoporosis, fatigue, type 1 diabetes, type 2 diabetes, prediabetes, metabolic syndrome, diabetic microangiopathy, diabetic nephropathy, hypothyroidism, breast cancer, prostate cancer, colon cancer, colorectal cancer, brain cancer, skin cancer, bladder cancer, stomach cancer, kidney cancer, esophageal cancer, pancreatic cancer, acute and chronic myeloid leukemia, multiple myeloma, myelodysplastic syndrome, head and neck cancer, bronchitis, cystic fibrosis, asthma, muscle soreness, wound healing, sepsis, cardiovascular disease, atherosclerosis, hypertension, myocarditis, heart failure, hypercholesterolemia, allergies, asthma, bronchitis, acute and chronic kidney disease, obesity, fatty liver disease, radiation dermatitis, periodontal disease, periodontitis, hemochromatosis, bacterial infections, oral submucous fibrosis, oral mucositis, oral lichen planus, gastritis, premenstrual syndrome, chronic obstructive pulmonary disease, chronic uveitis, familial adenomatous polyposis, H. pylori infection, chronic anterior uveitis, recurrent uveitis, ulcerative proctitis, beta-thalassemia, biliary dyskinesia, cholecystitis, transplant rejection, gingivitis, nephritis, premenstrual syndrome, thalassemia, pain, dyslipidemia, proteinuria, vascular stiffness, and the like, as well as any combinations thereof.

### EXAMPLES

Examples are provided hereinbelow. However, the present disclosure is to be understood to not be limited in its application to the specific experimentation, results, and laboratory procedures disclosed herein after. Rather, the Examples are simply provided as one of various embodiments and are meant to be exemplary, not exhaustive.

### Example 1

### Methods of Optimizing Production of CD-Modified Curcumin Compounds

As stated herein above, methods of increasing bioavailability and stability of an active agent for administration to a patient are commonly utilized in the art. One currently used method to increase bioavailability includes the use of a cyclodextrin, in which the active agent becomes bound, entrapped, and/or complexed within the central pocket of the cyclodextrin to increase the water solubility and stability of the substance.

However, for most active agents, current methods of manufacturing cyclodextrin complexes were not optimized for a given agent. Instead, established, generic methods have been employed, resulting in limited clinical potential. The present disclosure has demonstrated that optimization of the variables associated with manufacturing cyclodextrin complexes can result in significantly improved clinical activity relative to established methods. In the present disclosure, the methods for manufacturing cyclodextrin complexes for a given agent were optimized by employing a clinically relevant biological assay (e.g., a preclinical model of human disease) to quantitate the impact of varying manufacturing parameters on the clinical activity of the cyclodextrin complex.

In these embodiments, a particular weight (in grams) of agent (such as, but not limited to, curcumin) was mixed with a particular weight (in grams) of a cyclodextrin in a volume (in mls) of a solvent, to provide a desired molar ratio of agent to cyclodextrin and a desired total concentration of reactants. The reaction was then stirred under constant temperature of a certain time period. Products were then dried (such as, but not limited to, by air drying, oven drying, or lyophilization). Dried products were ground into powder with a mortar and pestle and stored at room temperature. Reconstitution was achieved by mixing the dried product in water or PBS.

Particular reaction parameters tested are detailed in Table 1. This Table contains the parameters that were varied and the relative clinical efficacy of the various methods tested in a mouse model of diabetes in order to create the optimized method for production of Cyclodextrin-Curcumin complexes.

Clinical activity of the product was then determined in a preclinical model.

### Activity of CD-Modified Curcumin as Studied in a Diabetes Model

Eight week old male C57BL/6 strain of mice were used. The mice were fed on standard diet and water ad libitum. Diabetes was induced by intraperitoneal injection of 60 mg/kg of a freshly prepared streptozotocin (STZ) dissolved in 0.1 M cold citrate buffer at a pH of 4.5. Body weight, food, and fluid intake were monitored daily. Induction was continued for 7 consecutive days until chronic polydipsia and polyuria was established. Post-prandial blood glucose levels were measured by blood glucose strips, and mice with levels higher than 400 mg/dL were defined as chronically diabetic and chosen for experiments. Test animals were treated with product by oral gavage. For a given product, n=6 animals were treated. Blood samples for whole blood glucose were obtained from the tail tip 4 hours post treatment, and blood glucose levels were measured by Roche blood glucose strips. Means and standard error of the means were calculated. Differences in mean post-prandial blood glucose 4 hours post treatment of animals treated with product and untreated control STX-induced diabetic animals (n=6) were calculated. Relative clinical effects of the products tested were determined by comparing the difference in blood glucose of treated relative to untreated controls.

The clinical activity results obtained for cyclodextrin-curcumin complexes in the preclinical model are shown in Table 1. Based on the results presented, it appears that all cyclodextrin-conjugated forms of curcumin had higher activity than curcumin alone. Moreover, in certain non-limiting embodiments, HPBCD-curcumin complexes may be more effective than SEBCD-, GCD-, or BCD-curcumin complexes. SEBCD-, GCD-, and BCD-curcumin complexes all gave similar results.

### Example 2

Comparison of the optimized CD-modification methods to established CD-modification methods

Table 2 contains particular reaction parameters tested and clinical activity results obtained in the preclinical model, respectively, for a comparison of the optimized methods of the present disclosure to established cyclodextrin modification methods and raw materials. As can be seen, the methods of the present disclosure produce a more active cyclodextrin-curcumin product. In particular, Table 2 demonstrates that the optimal method of the present disclosure has higher activity than currently available published methods. In addition, the betacyclodextrin-curcumin conjugate produced using the optimal method described herein has higher activity than curcumin alone and betacyclodextrin alone.

Table 3 contains a comparison of the clinical activity of the betacyclodextrin-curcumin complex produced by the optimized method disclosed herein relative to established diabetes treatments. As can be seen, the effects of betacyclodextrin-curcumin produced in accordance with the present disclosure are comparable to established diabetes treatments such as (but not limited to) glipizide, metformin, and insulin.

### Example 3

### CD-Modified Rutin Compounds Created by Various Methods

Table 4 lists various CD-modified rutin compounds that have been generated, along with the reaction conditions utilized and the variables assessed in each production method. In particular, Table 4 provides reaction conditions for the production of β-cyclodextrin-rutin complexes (BCD-rutin) where molar ratios were varied using a method developed on the curcumin diabetes studies of Example 1; reaction conditions for the production of β-cyclodextrin-rutin complexes (BCD-rutin) using the published “Kneading” method (i.e., high reactant concentrations, different solvent, and different reaction time and temperature than were used in the curcumin studies of Example 1); reaction conditions for the production of β-cyclodextrin-rutin complexes (BCD-rutin) using various solvents, various molar ratios, and various reaction times; reaction conditions for the production of β-cyclodextrin-rutin complexes (BCD-rutin) using various reactant concentrations, various molar ratios, various drying methods, and various reaction times; reaction conditions for the production of β-cyclodextrin-rutin complexes (BCD-rutin) using higher temperature and pressure; and reaction conditions for the production of β-cyclodextrin-rutin complexes (BCD-rutin) using higher temperature at various reactant concentrations and reaction times.

Table 5 illustrates the production of cyclodextrin-rutin complexes using different cyclodextrin compounds. These complexes include hydroxypropyl-beta-cyclodextrin-rutin complexes (HPBCD-rutin), hydroxyethyl beta-cyclodextrin-rutin complexes (HEBCD-rutin), and sulfobutyl-beta cyclodextrin-rutin complexes (SBBCD-rutin).

TABLE 1
Optimization of Cyclodextrin-Curcumin Complex Production Methods

| | | | | | | | | | | | |
|---|---|---|---|---|---|---|---|---|---|---|---|
| | Dose of | | | | | | | | | | |
| | Gavage | Difference | | | | | | | | | |
| | Diabetes | in 4 h Blood | Curcumin | CD | Molar | Rxn | Reactant | Rxn Temp | | | |
| Sample | Model | Glucose vs. | weight | weight | Ratio | Vol | Conc. | (° C.) & time | | Drying | |
| code | mg/kg | Untreated* | (g) | (g) | CD:Curc. | (mls) | (g/ml) | (h) | Solvent | method | Pressure |

Betacyclodextrin (BCD)
Molar ratios varied at low reactant concentration

| | | | | | | | | | | | |
|---|---|---|---|---|---|---|---|---|---|---|---|
| 1 | 25 | 123.09 | 1.60 | 10.00 | 2:1 | 80.00 | 0.15 | 40° C./18 h | 95% EtOH | Air dry | Atmospheric |
| 2 | 25 | 134.57 | 1.60 | 15.00 | 3:1 | 80.00 | 0.21 | 40° C./18 h | 95% EtOH | Air dry | Atmospheric |
| 3 | 25 | 139.76 | 1.60 | 20.00 | 4:1 | 80.00 | 0.27 | 40° C./18 h | 95% EtOH | Air dry | Atmospheric |
| 4 | 25 | 112.55 | 1.60 | 25.00 | 5:1 | 80.00 | 0.33 | 40° C./18 h | 95% EtOH | Air dry | Atmospheric |

Molar ratios varied at higher reactant concentration

| | | | | | | | | | | | |
|---|---|---|---|---|---|---|---|---|---|---|---|
| 5 | 25 | 136.64 | 3.20 | 10.00 | 1:1 | 80.00 | 0.17 | 40° C./18 h | 95% EtOH | Air dry | Atmospheric |
| 6 | 25 | 159.59 | 3.20 | 20.00 | 2:1 | 80.00 | 0.29 | 40° C./18 h | 95% EtOH | Air dry | Atmospheric |
| 7 | 25 | 145.42 | 3.20 | 30.00 | 3:1 | 80.00 | 0.42 | 40° C./18 h | 95% EtOH | Air dry | Atmospheric |

Repeat of molar ratios varied at higher reactant concentration

| | | | | | | | | | | | |
|---|---|---|---|---|---|---|---|---|---|---|---|
| 8 | 25 | 129.28 | 3.20 | 10.00 | 1:1 | 80.00 | 0.17 | 40° C./18 h | 95% EtOH | Air dry | Atmospheric |
| 9 | 25 | 148.43 | 3.20 | 20.00 | 2:1 | 80.00 | 0.29 | 40° C./18 h | 95% EtOH | Air dry | Atmospheric |
| 10 | 25 | 137.49 | 3.20 | 30.00 | 3:1 | 80.00 | 0.42 | 40° C./18 h | 95% EtOH | Air dry | Atmospheric |
| 11 | 25 | 116.11 | 3.20 | 40.00 | 4:1 | 80.00 | 0.54 | 40° C./18 h | 95% EtOH | Air dry | Atmospheric |

Reactant concentration varied at 1:1 molar ratio

| | | | | | | | | | | | |
|---|---|---|---|---|---|---|---|---|---|---|---|
| 12 | 250 | 130.2 | 0.40 | 1.20 | 1:1 | 20.00 | 0.08 | 40° C./6 h | 95% EtOH | Air dry | Atmospheric |
| 13 | 250 | 138.9 | 0.60 | 1.80 | 1:1 | 20.00 | 0.12 | 40° C./6 h | 95% EtOH | Air dry | Atmospheric |
| 14 | 250 | 153.1 | 0.80 | 2.50 | 1:1 | 20.00 | 0.17 | 40° C./6 h | 95% EtOH | Air dry | Atmospheric |
| 15 | 250 | 149.7 | 1.00 | 3.10 | 1:1 | 20.00 | 0.21 | 40° C./6 h | 95% EtOH | Air dry | Atmospheric |
| 16 | 250 | 150.6 | 1.20 | 3.70 | 1:1 | 20.00 | 0.25 | 40° C./6 h | 95% EtOH | Air dry | Atmospheric |
| 17 | 250 | 155.9 | 1.40 | 4.30 | 1:1 | 20.00 | 0.29 | 40° C./6 h | 95% EtOH | Air dry | Atmospheric |

Reactant concentration varied at 2:1 molar ratio

| | | | | | | | | | | | |
|---|---|---|---|---|---|---|---|---|---|---|---|
| 18 | 250 | 141.04 | 0.40 | 2.50 | 2:1 | 20.00 | 0.15 | 40° C./6 h | 95% EtOH | Air dry | Atmospheric |
| 19 | 250 | 150.23 | 0.60 | 3.70 | 2:1 | 20.00 | 0.22 | 40° C./6 h | 95% EtOH | Air dry | Atmospheric |
| 20 | 250 | 165.51 | 1.00 | 6.20 | 2:1 | 20.00 | 0.36 | 40° C./6 h | 95% EtOH | Air dry | Atmospheric |
| 21 | 250 | 163.9 | 1.20 | 7.40 | 2:1 | 20.00 | 0.43 | 40° C./6 h | 95% EtOH | Air dry | Atmospheric |
| 22 | 250 | 168.75 | 1.40 | 8.60 | 2:1 | 20.00 | 0.50 | 40° C./6 h | 95% EtOH | Air dry | Atmospheric |

Reactant concentration varied at 2:1 molar ratio, 6 hour mixing, 5 mg/kg dose

| | | | | | | | | | | | |
|---|---|---|---|---|---|---|---|---|---|---|---|
| 23 | 5 | 70.58 | 0.40 | 2.50 | 2:1 | 20.00 | 0.15 | 40° C./6 h | 95% EtOH | Air dry | Atmospheric |
| 24 | 5 | 97.62 | 1.60 | 10.00 | 2:1 | 20.00 | 0.58 | 40° C./6 h | 95% EtOH | Air dry | Atmospheric |
| 25 | 5 | 109.69 | 2.40 | 15.00 | 2:1 | 20.00 | 0.87 | 40° C./6 h | 95% EtOH | Air dry | Atmospheric |

Reactant concentration varied at 2:1 molar ratio, 18 hour mixing, 5 mg/kg dose

| | | | | | | | | | | | |
|---|---|---|---|---|---|---|---|---|---|---|---|
| 26 | 5 | 80.47 | 0.40 | 2.50 | 2:1 | 20.00 | 0.15 | 40° C./18 h | 95% EtOH | Air dry | Atmospheric |
| 27 | 5 | 108.35 | 1.60 | 10.00 | 2:1 | 20.00 | 0.58 | 40° C./18 h | 95% EtOH | Air dry | Atmospheric |
| 28 | 5 | 117.8 | 2.40 | 15.00 | 2:1 | 20.00 | 0.87 | 40° C./18 h | 95% EtOH | Air dry | Atmospheric |

Reactant concentration varied at 2:1 molar ratio, 18 hour mixing, 25 mg/kg dose

| | | | | | | | | | | | |
|---|---|---|---|---|---|---|---|---|---|---|---|
| 29 | 25 | 166.86 | 3.20 | 20.00 | 2:1 | 80.00 | 0.29 | 40° C./18 h | 95% EtOH | Air dry | Atmospheric |
| 30 | 25 | 163.54 | 4.00 | 25.00 | 2:1 | 80.00 | 0.36 | 40° C./18 h | 95% EtOH | Air dry | Atmospheric |
| 31 | 25 | 148.45 | 5.60 | 35.00 | 2:1 | 80.00 | 0.51 | 40° C./18 h | 95% EtOH | Air dry | Atmospheric |

Repeat of reactant concentration varied at 2:1 molar ratio, 18 hour mixing, 25 mg/kg dose

| | | | | | | | | | | | |
|---|---|---|---|---|---|---|---|---|---|---|---|
| 32 | 25 | 152.89 | 3.20 | 20.00 | 2:1 | 80.00 | 0.29 | 40° C./18 h | 95% EtOH | Air dry | Atmospheric |
| 33 | 25 | 155.54 | 4.00 | 25.00 | 2:1 | 80.00 | 0.36 | 40° C./18 h | 95% EtOH | Air dry | Atmospheric |
| 34 | 25 | 143.06 | 4.80 | 30.00 | 2:1 | 80.00 | 0.44 | 40° C./18 h | 95% EtOH | Air dry | Atmospheric |
| 35 | 25 | 134.68 | 5.60 | 35.00 | 2:1 | 80.00 | 0.51 | 40° C./18 h | 95% EtOH | Air dry | Atmospheric |

Time varied at optimal molar ratio and reactant concentration

| | | | | | | | | | | | |
|---|---|---|---|---|---|---|---|---|---|---|---|
| 36 | 25 | 126.99 | 3.20 | 20.00 | 2:1 | 80.00 | 0.29 | 40° C./0 h | 95% EtOH | Air dry | Atmospheric |
| 37 | 25 | 155.33 | 3.20 | 20.00 | 2:1 | 80.00 | 0.29 | 40° C./18 h | 95% EtOH | Air dry | Atmospheric |
| 38 | 25 | 164.16 | 3.20 | 20.00 | 2:1 | 80.00 | 0.29 | 40° C./72 h | 95% EtOH | Air dry | Atmospheric |

Hydroxypropylbeta Cyclodextrin (HPBCD)
From above, partial information, may be redundant with below or need filling in

| | | | | | | | | | | | |
|---|---|---|---|---|---|---|---|---|---|---|---|
| 1 | 25 | 173.71 | 1.60 | 10.00 | | | | | | | |
| 2 | 25 | 100.8 | 3.20 | 20.00 | 2:1 | 80.00 | 0.29 | 40° C./18 h | 95% EtOH | Air dry | Atmospheric |

Molar ratios varied at low reactant concentration

| | | | | | | | | | | | |
|---|---|---|---|---|---|---|---|---|---|---|---|
| 3 | 25 | 174.48 | 1.6 | 6.7 | 1:1 | 80.00 | 0.10 | 40° C./18 h | 95% EtOH | Air dry | Atmospheric |
| 4 | 25 | 183.42 | 1.6 | 13.4 | 2:1 | 80.00 | 0.19 | 40° C./18 h | 95% EtOH | Air dry | Atmospheric |
| 5 | 25 | 189.34 | 1.6 | 20.1 | 3:1 | 80.00 | 0.27 | 40° C./18 h | 95% EtOH | Air dry | Atmospheric |
| 6 | 25 | 189.35 | 1.6 | 26.8 | 4:1 | 80.00 | 0.36 | 40° C./18 h | 95% EtOH | Air dry | Atmospheric |

Molar ratios varied at higher reactant concentration

| | | | | | | | | | | | |
|---|---|---|---|---|---|---|---|---|---|---|---|
| 7 | 25 | 186.06 | 3.2 | 13.4 | 1:1 | 80.00 | 0.21 | 40° C./18 h | 95% EtOH | Air dry | Atmospheric |
| 8 | 25 | 194.18 | 3.2 | 26.8 | 2:1 | 80.00 | 0.38 | 40° C./18 h | 95% EtOH | Air dry | Atmospheric |
| 9 | 25 | 203.69 | 3.2 | 40.2 | 3:1 | 80.00 | 0.54 | 40° C./18 h | 95% EtOH | Air dry | Atmospheric |
| 10 | 25 | 185.79 | 3.2 | 53.6 | 4:1 | 80.00 | 0.71 | 40° C./18 h | 95% EtOH | Air dry | Atmospheric |
| 11 | 25 | 177.47 | 3.2 | 67 | 5:1 | 80.00 | 0.88 | 40° C./18 h | 95% EtOH | Air dry | Atmospheric |

Molar ratios varied and reactant concentration varied

| | | | | | | | | | | | |
|---|---|---|---|---|---|---|---|---|---|---|---|
| 12 | 25 | 188.53 | 3.2 | 26.8 | 1:1 | 80.00 | 0.38 | 40° C./18 h | 95% EtOH | Air dry | Atmospheric |
| 13 | 25 | 192.86 | 3.2 | 40.2 | 3:1 | 80.00 | 0.54 | 40° C./18 h | 95% EtOH | Air dry | Atmospheric |
| 14 | 25 | 186.33 | 4 | 33.5 | 3:1 | 80.00 | 0.47 | 40° C./18 h | 95% EtOH | Air dry | Atmospheric |
| 15 | 25 | 194.09 | 4 | 44.8 | 1:1 | 80.00 | 0.61 | 40° C./18 h | 95% EtOH | Air dry | Atmospheric |
| 16 | 25 | 191.21 | 4.8 | 40.2 | 2:1 | 80.00 | 0.56 | 40° C./18 h | 95% EtOH | Air dry | Atmospheric |
| 17 | 25 | 199.97 | 4.8 | 60.3 | 2:1 | 80.00 | 0.81 | 40° C./18 h | 95% EtOH | Air dry | Atmospheric |

Gamma cyclodextrin (GCD)
Molar ratios varied

| | | | | | | | | | | | |
|---|---|---|---|---|---|---|---|---|---|---|---|
| 1 | 25 | 144.17 | 3.2 | 11.3 | 1:1 | 80.00 | 0.18 | 40° C./18 h | 95% EtOH | Air dry | Atmospheric |
| 2 | 25 | 157.33 | 3.2 | 22.6 | 2:1 | 80.00 | 0.32 | 40° C./18 h | 95% EtOH | Air dry | Atmospheric |
| 3 | 25 | 145.05 | 3.2 | 33.9 | 3:1 | 80.00 | 0.46 | 40° C./18 h | 95% EtOH | Air dry | Atmospheric |
| 4 | 25 | 153.9 | 3.2 | 45.2 | 4:1 | 80.00 | 0.61 | 40° C./18 h | 95% EtOH | Air dry | Atmospheric |
| 5 | 25 | 131.29 | 3.2 | 56.5 | 5:1 | 80.00 | 0.75 | 40° C./18 h | 95% EtOH | Air dry | Atmospheric |

Concentration varied for 2:1 molar ratio

| | | | | | | | | | | | |
|---|---|---|---|---|---|---|---|---|---|---|---|
| 6 | 25 | 163.89 | 1.6 | 5.6 | 2:1 | 80.00 | 0.09 | 40° C./18 h | 95% EtOH | Air dry | Atmospheric |

Sulfobutyl ether-β-cyclodextrin (SEBCD)
Molar ratios varied

| | | | | | | | | | | | |
|---|---|---|---|---|---|---|---|---|---|---|---|
| 1 | 25 | 144.62 | 3.2 | 11.2 | 1:1 | 80.00 | 1.85 | 40° C./18 h | 95% EtOH | Air dry | Atmospheric |
| 2 | 25 | 161.43 | 3.2 | 22.4 | 2:1 | 80.00 | 2.06 | 40° C./18 h | 95% EtOH | Air dry | Atmospheric |
| 3 | 25 | 157.59 | 3.2 | 33.6 | 3:1 | 80.00 | 2.01 | 40° C./18 h | 95% EtOH | Air dry | Atmospheric |
| 4 | 25 | 104.51 | 3.2 | 44.8 | 4:1 | 80.00 | 1.35 | 40° C./18 h | 95% EtOH | Air dry | Atmospheric |
| 5 | 25 | 96.85 | 3.2 | 56 | 5:1 | 80.00 | 1.25 | 40° C./18 h | 95% EtOH | Air dry | Atmospheric |

Unconjugated Curcumin

| | | | | | | | | | | | |
|---|---|---|---|---|---|---|---|---|---|---|---|
| 1 | 5 | 29.8 | NA | NA | NA | NA | NA | NA | NA | NA | NA |
| 2 | 25 | 60.22 | NA | NA | NA | NA | NA | NA | NA | NA | NA |

*NA—Not Applicable

TABLE 2
Comparison of the Clinical Activity of the Optimized Method
Relative to Established Methods and Raw Materials

| | |
|---|---|
| Dose of Gavage | Difference in 4 h |
| Diabetes Model | Blood Glucose vs. |
| mg/kg | Untreated* |

Optimized Method Curcumin Betacyclodextrin complex formation (Average of 3 independent syntheses)*

| | | |
|---|---|---|
| Synthesis 1 | 25 | 154.7 |
| Synthesis 2 | 25 | 166.9 |
| Synthesis 3 | 25 | 150.89 |
| Average of 3 syntheses | | 157.5 |
| Established Methods | | |
| Solvent Evaporation Method 1:1 molar ratio Betacyclodextrin to curcumin | 25 | 106.41 |
| Solvent Evaporation Method 2:1 molar ratio Betacyclodextrin to curcumin | 25 | 111.45 |
| Kneading Method 1:1 molar ratio Betacyclodextrin to curcumin | 25 | 134.52 |
| Kneading Method 2:1 molar ratio Betacyclodextrin to curcumin | 25 | 146.13 |
| Methanol reflux Method | 25 | 103.35 |
| Lyophilization method | 25 | 112.73 |
| Pulverization method | 25 | 97.11 |
| Acetone Method | 25 | 123.64 |
| Co-solvent Method | 25 | 111.25 |
| Freeze Drying Filtration Method BCD-CUR | 25 | 110.93 |
| Freeze Drying Method 1:1 molar ratio Betacyclodextrin to curcumin | 25 | 117.27 |
| Freeze Drying Method 2:1 molar ratio Betacyclodextrin to curcumin | 25 | 101.86 |
| Uncomplexed raw materials | | |
| Curcumin alone | 25 | 60.22 |
| Betacyclodextrin alone | 25 | 35.46 |

*Optimal method conditions: curcumin conc 3.20 g, betacyclodextrin 20 g, 2:1 molar ratio, 80 mls, 40 C., 95% EtOH, 18 h mixing time, air dry under atmospheric conditions

TABLE 3
Comparison of the Clinical Activity of the Optimized
Method Relative to Established Diabetes Treatments

| | |
|---|---|
| Dose of Gavage | Difference in 4 h |
| Diabetes Model | Blood Glucose vs. |
| mg/kg | Untreated* |

Optimized Method Curcumin Betacyclodextrin complex
formation (Average of 3 independent syntheses)*

| Synthesis 1 | 25 | | 154.7 |
|---|---|---|---|
| Synthesis 2 | 25 | | 166.9 |
| Synthesis 3 | 25 | | 150.89 |
| Average of 3 syntheses | | | 157.5 |
| Clinical activity of | | | |
| established diabetes treatments | | | |
| Glipizide-5-oral | 5 | | 198.0 |
| Metformin-200-oral | 200 | | 173.9 |
| Insulin-1 IU-sc | (1 | IU) | 236.2 |
| Insulin-0.5 IU-sc | (0.5 | IU) | 203.2 |

| | | |
|---|---|---|
| Metformin-250-oral | 250 | 181.7 |

TABLE 4
Optimization of β-Cyclodextrin-Rutin Complex Production Methods

| | | | | | | | | | |
|---|---|---|---|---|---|---|---|---|---|
| | Rutin | CD | | Reaction | Reaction | | | | |
| Sample | weight | weight | Molar Ratio | volume | Temp | | Reaction | Drying | |
| code | (g) | (g) | CD:rutin | (mls) | (° C.) | Solvent | time (h) | method | Pressure |

Production of β-Cyclodextrin-Rutin Complexes Using Various Molar Ratios

| | | | | | | | | | |
|---|---|---|---|---|---|---|---|---|---|
| 1 | 3.2 | 5.8 | 1:1 | 80 | 40 | 95% EtOH | 18 | Air dry | Atmospheric |
| 2 | 3.2 | 11.6 | 2:1 | 80 | 40 | 95% EtOH | 18 | Air dry | Atmospheric |
| 3 | 3.2 | 17.3 | 3:1 | 80 | 40 | 95% EtOH | 18 | Air dry | Atmospheric |

Production of β-Cyclodextrin-Rutin Complexes Using Prior Art “Kneading” Method

| | | | | | | | | | |
|---|---|---|---|---|---|---|---|---|---|
| 4 | 15 | 54.3 | 2:1 | 35 | 20 | 33% EtOH | 0.5 | Oven dried | Atmospheric |

Production of β-Cyclodextrin-Rutin Complexes Using Various Solvents, Molar Ratios, and Reaction Times

| | | | | | | | | | |
|---|---|---|---|---|---|---|---|---|---|
| 5 | 3.2 | 5.8 | 1:1 | 80 | 40 | Water | 18 | Lyophilized | Atmospheric |
| 6 | 3.2 | 11.6 | 2:1 | 80 | 40 | Water | 18 | Lyophilized | Atmospheric |
| 7 | 3.2 | 5.8 | 1:1 | 80 | 40 | 1:1 Water 95% EtOH | 18 | Lyophilized | Atmospheric |
| 8 | 3.2 | 11.6 | 2:1 | 80 | 40 | 1:1 Water 95% EtOH | 18 | Lyophilized | Atmospheric |
| 9 | 3.2 | 5.8 | 1:1 | 80 | 40 | 2:1 Water 95% EtOH | 18 | Lyophilized | Atmospheric |
| 10 | 3.2 | 11.6 | 2:1 | 80 | 40 | 2:1 Water 95% EtOH | 18 | Lyophilized | Atmospheric |
| 11 | 3.2 | 5.8 | 1:1 | 80 | 40 | Water | 6 | Lyophilized | Atmospheric |
| 12 | 3.2 | 11.6 | 2:1 | 80 | 40 | Water | 6 | Lyophilized | Atmospheric |
| 13 | 3.2 | 5.8 | 1:1 | 80 | 40 | 1:1 Water 95% EtOH | 6 | Lyophilized | Atmospheric |
| 14 | 3.2 | 11.6 | 2:1 | 80 | 40 | 1:1 Water 95% EtOH | 6 | Lyophilized | Atmospheric |
| 15 | 3.2 | 5.8 | 1:1 | 80 | 40 | 2:1 Water 95% EtOH | 6 | Lyophilized | Atmospheric |
| 16 | 3.2 | 11.6 | 2:1 | 80 | 40 | 2:1 Water 95% EtOH | 6 | Lyophilized | Atmospheric |

Production of β-Cyclodextrin-Rutin Complexes Using Various Reaction Conditions

| | | | | | | | | | |
|---|---|---|---|---|---|---|---|---|---|
| 17 | 96 | 174.0 | 1:1 | 80 | 20 | 95% EtOH | 1 | Oven dried | Atmospheric |
| 18 | 96 | 174.0 | 1:1 | 80 | 20 | 95% EtOH | 1 | Lyophilized | Atmospheric |
| 19 | 96 | 174.0 | 1:1 | 80 | 20 | Water | 1 | Oven dried | Atmospheric |
| 20 | 96 | 174.0 | 1:1 | 80 | 20 | Water | 1 | Lyophilized | Atmospheric |
| 21 | 96 | 174.0 | 1:1 | 80 | 20 | 1:1 Water 95% EtOH | 1 | Oven dried | Atmospheric |
| 22 | 96 | 174.0 | 1:1 | 80 | 20 | 1:1 Water 95% EtOH | 1 | Lyophilized | Atmospheric |
| 23 | 96 | 174.0 | 2:1 | 80 | 20 | 2:1 Water 95% EtOH | 1 | Oven dried | Atmospheric |
| 24 | 96 | 174.0 | 2:1 | 80 | 20 | 2:1 Water 95% EtOH | 1 | Lyophilized | Atmospheric |
| 25 | 96 | 174.0 | 1:1 | 80 | 20 | 95% EtOH | 6 | Oven dried | Atmospheric |
| 26 | 96 | 174.0 | 1:1 | 80 | 20 | 95% EtOH | 6 | Lyophilized | Atmospheric |
| 27 | 96 | 174.0 | 1:1 | 80 | 20 | Water | 6 | Oven dried | Atmospheric |
| 28 | 96 | 174.0 | 1:1 | 80 | 20 | Water | 6 | Lyophilized | Atmospheric |
| 29 | 96 | 174.0 | 1:1 | 80 | 20 | 1:1 Water 95% EtOH | 6 | Oven dried | Atmospheric |
| 30 | 96 | 174.0 | 1:1 | 80 | 20 | 1:1 Water 95% EtOH | 6 | Lyophilized | Atmospheric |
| 31 | 96 | 174.0 | 1:1 | 80 | 20 | 2:1 Water 95% EtOH | 6 | Oven dried | Atmospheric |
| 32 | 96 | 174.0 | 1:1 | 80 | 20 | 2:1 Water 95% EtOH | 6 | Lyophilized | Atmospheric |
| 33 | 96 | 348 | 2:1 | 80 | 20 | 95% EtOH | 1 | Oven dried | Atmospheric |
| 34 | 96 | 348.0 | 2:1 | 80 | 20 | 95% EtOH | 1 | Lyophilized | Atmospheric |
| 35 | 96 | 348.0 | 2:1 | 80 | 20 | Water | 1 | Oven dried | Atmospheric |
| 36 | 96 | 348.0 | 2:1 | 80 | 20 | Water | 1 | Lyophilized | Atmospheric |
| 37 | 96 | 348 | 2:1 | 80 | 20 | 1:1 Water 95% EtOH | 1 | Oven dried | Atmospheric |
| 38 | 96 | 348 | 2:1 | 80 | 20 | 1:1 Water 95% EtOH | 1 | Lyophilized | Atmospheric |
| 39 | 96 | 348 | 2:1 | 80 | 20 | 2:1 Water 95% EtOH | 1 | Oven dried | Atmospheric |
| 40 | 96 | 348 | 2:1 | 80 | 20 | 2:1 Water 95% EtOH | 1 | Lyophilized | Atmospheric |
| 41 | 96 | 348 | 2:1 | 80 | 20 | 95% EtOH | 6 | Oven dried | Atmospheric |
| 42 | 96 | 348 | 2:1 | 80 | 20 | 95% EtOH | 6 | Lyophilized | Atmospheric |
| 43 | 96 | 348 | 2:1 | 80 | 20 | Water | 6 | Oven dried | Atmospheric |
| 44 | 96 | 348 | 2:1 | 80 | 20 | Water | 6 | Lyophilized | Atmospheric |
| 45 | 96 | 348 | 2:1 | 80 | 20 | 1:1 Water 95% EtOH | 6 | Oven dried | Atmospheric |
| 46 | 96 | 348 | 2:1 | 80 | 20 | 1:1 Water 95% EtOH | 6 | Lyophilized | Atmospheric |
| 47 | 96 | 348 | 2:1 | 80 | 20 | 2:1 Water 95% EtOH | 6 | Oven dried | Atmospheric |
| 48 | 96 | 348 | 2:1 | 80 | 20 | 2:1 Water 95% EtOH | 6 | Lyophilized | Atmospheric |
| 49 | 96 | 174 | 1:1 | 80 | 20 | 1:1 Water 95% EtOH | 10 | Oven dried | Atmospheric |
| 50 | 96 | 174 | 1:1 | 80 | 20 | 2:1 Water 95% EtOH | 10 | Oven dried | Atmospheric |

Production of β-Cyclodextrin-Rutin Complexes Using Higher Temperature and Pressure

| | | | | | | | | | |
|---|---|---|---|---|---|---|---|---|---|
| 51 | 3.2 | 5.8 | 1:1 | 80 | 121 | 1:1 Water 95% EtOH | 0.5 | Oven dried | 30 psi (15 psi |
| | | | | | | | | | above atmospheric) |
| 52 | 356 | 644 | 1:1 | 600 | 121 | 1:1 Water 95% EtOH | 0.5 | Oven dried | 30 psi (15 psi |
| | | | | | | | | | above atmospheric) |
| 53 | 4 | 6 | 1:1 | 6 | 121 | 1:1 Water 95% EtOH | 0.5 | Oven dried | 30 psi (15 psi |
| | | | | | | | | | above atmospheric) |
| 54 | 3.2 | 5.8 | 1:1 | 80 | 80 | 1:1 Water 95% EtOH | 1 | Oven dried | Atmospheric |

Production of β-Cyclodextrin-Rutin Complexes Using Higher Temperature
at Various Reactant Concentrations and Reaction Times

| | | | | | | | | | |
|---|---|---|---|---|---|---|---|---|---|
| 55 | 3.2 | 5.8 | 1:1 | 80 | 80 | 1:1 Water 95% EtOH | 6 | Oven dried | Atmospheric |
| 56 | 96 | 174 | 1:1 | 80 | 20 | 1:1 Water 95% EtOH | 10 | Oven dried | Atmospheric |

Unconjugated Rutin

| | | | | | | | | | |
|---|---|---|---|---|---|---|---|---|---|
| 57 | NA | NA | NA | NA | NA | NA | NA | NA | NA |

*NA—Not Applicable

TABLE 5
Production of Cyclodextrin-Rutin Complexes with Different Cyclodextrin Compounds

| | | | | | | | | | |
|---|---|---|---|---|---|---|---|---|---|
| | Rutin | CD | | Reaction | Reaction | | | | |
| Sample | weight | weight | Molar Ratio | volume | Temp | | Reaction | Drying | |
| code | (g) | (g) | CD:rutin | (mls) | (° C.) | Solvent | time (h) | method | Pressure |

Production of γ-Cyclodextrin-Rutin Complexes

| | | | | | | | | | |
|---|---|---|---|---|---|---|---|---|---|
| 1 | 3.2 | 6.6 | 1:1 | 80 | 40 | 1:1 Water 95% EtOH | 18 | Oven dried | Atmospheric |
| 2 | 3.2 | 6.6 | 1:1 | 80 | 40 | 2:1 Water 95% EtOH | 18 | Oven dried | Atmospheric |

Production of Hydroxypropyl-γ-Cyclodextrin-Rutin Complexes

| | | | | | | | | | |
|---|---|---|---|---|---|---|---|---|---|
| 3 | 3.2 | 9 | 1:1 | 80 | 40 | 1:1 Water 95% EtOH | 18 | Oven dried | Atmospheric |
| 4 | 3.2 | 9 | 1:1 | 80 | 40 | 2:1 Water 95% EtOH | 18 | Oven dried | Atmospheric |

Production of α-Cyclodextrin-Rutin Complexes

| | | | | | | | | | |
|---|---|---|---|---|---|---|---|---|---|
| 5 | 3.2 | 5 | 1:1 | 80 | 40 | 1:1 Water 95% EtOH | 18 | Oven dried | Atmospheric |

Production of HPBCD-Rutin Complexes

| | | | | | | | | | |
|---|---|---|---|---|---|---|---|---|---|
| 6 | 3.2 | 7.8 | 1:1 | 80 | 40 | 95% EtOH | 18 | Air dry | Atmospheric |
| 7 | 3.2 | 15.7 | 2:1 | 80 | 40 | 95% EtOH | 18 | Air dry | Atmospheric |
| 8 | 3.2 | 23.5 | 3:1 | 80 | 40 | 95% EtOH | 18 | Air dry | Atmospheric |

Production of HEBCD-Rutin

| | | | | | | | | | |
|---|---|---|---|---|---|---|---|---|---|
| 9 | 3.2 | 7.3 | 1:1 | 80 | 40 | 1:1 Water 95% EtOH | 18 | Oven dried | Atmospheric |

Production of SBBCD-Rutin

| | | | | | | | | | |
|---|---|---|---|---|---|---|---|---|---|
| 10 | 3.2 | 7.4 | 1:1 | 80 | 40 | 1:1 Water 95% EtOH | 18 | Oven dried | Atmospheric |

Unconjugated Rutin

| | | | | | | | | | |
|---|---|---|---|---|---|---|---|---|---|
| 11 | NA | NA | NA | NA | NA | NA | NA | NA | NA |

*NA—Not Applicable

### Example 4

Many currently available anti-viral agents are being studied to determine their effects on SARS-COV-2 infection. However, only limited efficacy of approved and experimental anti-viral drugs has been observed. Thus far, no therapeutic agents have been identified that prevent or treat SARS-COV-2 infection. In addition, there are limits on various treatment modalities because of the toxic and lethal effects of the Pathogenic Host Response (PHR). For example, the acute respiratory distress syndrome (ARDS) observed in some COVID-19 patients is akin to an autoinflammatory disease or septic shock. Inflammation, fibrosis, endothelial damage, vascular leakage, coagulation, and alveolar wall thickening have all observed in COVID-19 patients. PHR is the primary cause of morbidity and mortality for COVID-19 patients due to inflammation-induced lung injury and organ failure.

Therefore, compositions and methods for modulating PHR, referred to herein as “anti-PHR agents,” were identified and selected as described in U.S. Ser. No. 63/234,902, filed Aug. 19, 2021. The anti-PHR agents selected have anti-inflammatory, anti-coagulant, anti-lung fibrotic, and/or anti-viral activity, ameliorate acute lung injury, in silico SARS-COV-2 protein binding, and are designated as GRAS (Generally Regarded As Safe). The anti-PHR agents have also been demonstrated to inhibit SARS-COV-2 replication in vitro and to be highly efficacious in murine COVID-19 model assays.

The anti-PHR agents were screened in a human ACE-2 transgenic COVID-19 mouse model. This model exhibits significant mortality, with approximately 40% mortality at Day 8. The model also exhibits significant morbidity and recapitulates human COVID-19 viral lung pathology, cytokine storm, hemorrhage, fibrosis, labored breathing, lethargy, and wasting disease. Morbidity/PHR can be measured using body weight; a weight loss of approximately 50% is observed in the model by Day 7.

These various anti-PHR agents would further benefit from being modified to increase the bioavailability thereof. As such, the present disclosure includes methods of modifying anti-PHR agents to provide increased bioavailability when compared to unmodified agent.

### Materials and Methods:

To compare the clinical response and activity of our candidates, the therapeutic response of these candidates against SARS-COV-2 was determined at various multiplicities of infection. Briefly, the SARS-COV-2 virus strain (obtained from CDC BEI Resources Repository) was propagated using Vero E6 cells. Stock virus was prepared after serial passages in Vero E6 cells in infection media. To evaluate the effect of the compounds in vitro, Vero E6 cells was pre-treated with compounds diluted in infection media for 1 hour prior to infection by SARS-COV-2 virus. Supernatants were collected at various time points (0, 2, 6, 12, 24, 48 and 72 hours) to quantify viral loads by quantitative real-time RT-PCR. The 50% effective concentrations (EC50) of the drug compounds that inhibit viral replication was evaluated.

Cytotoxicity in VeroE6 cells was assessed using the viability assay by the Cell Counting Kit-8 (CCK8) method. Following treatments as mentioned above, cells were treated with 5 mg/ml CCK8 and incubated for 2 hours. The OD value was read at 570 nm on a microplate reader. Cell viability was calculated as the percent ratio of absorbance of the samples against untreated controls.

The SARS-Cov-2 in vivo model was developed using intranasal infections of SARS-COV-2 strain in hACE2 transgenic mice. A vector carrying a human ACE2-coding sequence was introduced into C57BL/6 mice which subsequently developed a successful hACE2 transgenic mouse strain. When infected with SARS-COV-2 virus, it caused a lethal pulmonary syndrome in mice. The titer of virus was determined by a plaque assay and mice were anaesthetized and infected intranasally with the indicated dosage of SARS-COV in DMEM. The severity of the infection can be adjusted based on the dose amount that was used. Mice infected with low amounts experienced transient weight loss and minor clinical signs of disease. Infection with higher amounts resulted in significant weight loss by day 5, significant clinical disease, including fever, hunched posture, decreased activity, labored breathing, and subsequently, death.

Treatments were administered at various doses. Negative and positive controls were used to compare therapeutic efficacy and benefit. Mice were weighed and observed for clinical signs daily throughout the study. Clinical changes were noted daily, and biological samples were collected at 0, 1-, 2-, 4-, and 7-days post-infection to allow for profiling of the different phases of infection. The virus load in the lung was quantified at the end of experiment. Morphology and histopathology of the lungs, heart, kidneys, and other organs were conducted and correlated to clinical results. The resolution of clinical symptoms and signs, viral titers, and histopathological parameters were evaluated and compared.

### Results:

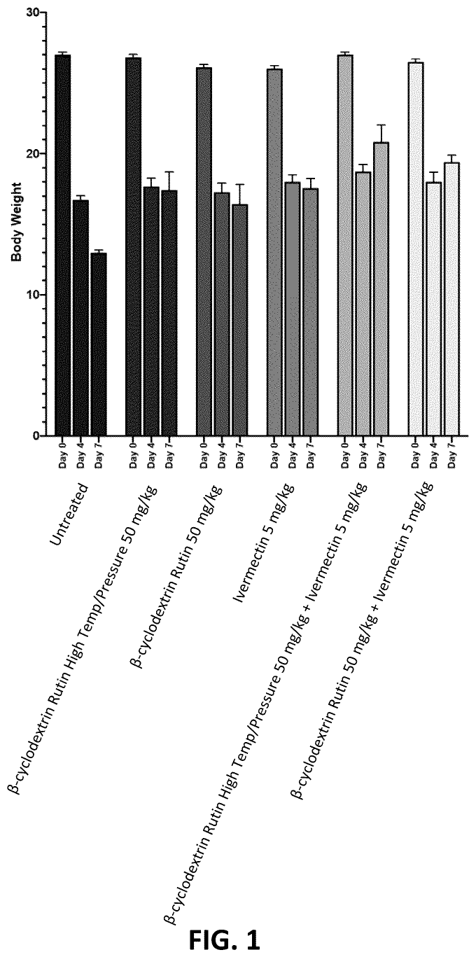

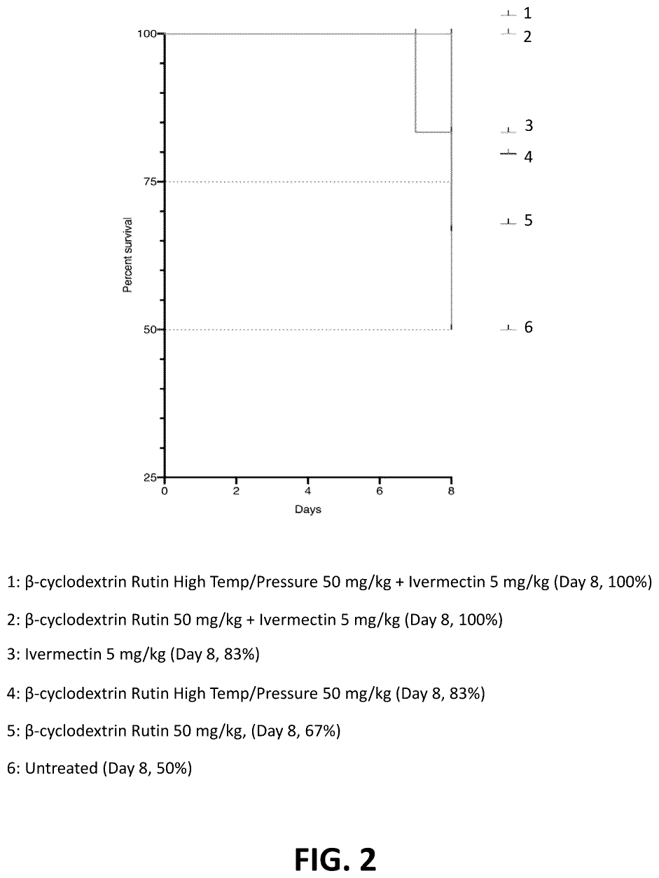

FIGS. 1-2 illustrate the results obtained with the mouse model when using a high bioavailability anti-PHR agent (BCD-rutin) in combination with an anti-viral agent (ivermectin) in the bimodal combinatorial therapy of the present disclosure. The high bioavailability BCD-rutin was prepared by two different ways: the conventional prior art method, or the methods described herein. The conventional prior art method involves preparing the BCD-rutin complex at room temperature and atmospheric pressure in a kitchen laid mixer (labeled in FIGS. 1-2 simply as “β-cyclodextrin Rutin”). In the novel production method described herein cyclodextrin and agent are incubated under conditions that include a temperature above room temperature (such as, but not limited to, a temperature in a range of from about 20° C. to about 300° C.) and a pressure above atmospheric pressure (such as, but not limited to, a pressure in a range of from about 15 PSI to about 200 PSI) for a sufficient amount of time (such as, but not limited to, a period in a range of from about 0.1 hours to about 18 hours) to form the cyclodextrin-modified agent (labeled in FIGS. 1-2 as “β-cyclodextrin Rutin High Temp/Pressure”).

As can be seen in FIG. 1, all of the treatment modalities had some effect on morbidity, with the two bimodal combinatorial therapies showing the greatest effect. Also, the addition of higher temperature and pressure to the production of the high bioavailability anti-PHR agent provided a more robust response on morbidity. The combinatorial therapies both exhibited weight gain at Day 7, with the combinatorial therapy that included BCD-rutin produced under higher temperature and pressure having a greater effect than the therapy that included BCD-rutin produced at room temperature and atmospheric pressure.

FIG. 2 demonstrates that, while a 50% mortality rate was observed in the untreated group, the mortality rate dropped to 17-33% (i.e., 67-83% survival rate) in the single modality treatment groups (i.e., ivermectin alone, BCD-rutin alone, BCD-rutin high temp/pressure alone). However, a 100% survival rate was observed in both of the combinatorial therapy treatment groups.

### Example 5

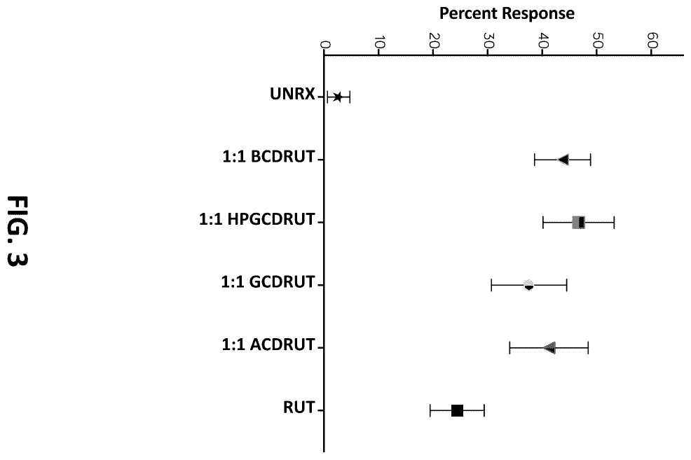

This Example contains an analysis of the anti-PHR agent rutin and various cyclodextrin-modified forms thereof against SARS-COV-2 and diabetes models as described previously. In this Example, rutin was modified with alpha-cyclodextrin (ACDRUT), beta-cyclodextrin (BCDRUT), gamma-cyclodextrin (GCDRUT), Hydroxypropylbetacyclodextrin (HPBCDRUT), and/or Hydroxypropylgammacyclodextrin (HPGCDRUT), and the ability of unmodified rutin and cyclodextrin-modified forms thereof to inhibit SARS-COV-2 replication in vitro was examined. As can be seen in FIG. 3, unmodified rutin inhibited SARS-COV-2 replication by approximately 25% over untreated. However, cyclodextrin modification of rutin resulted in a synergistic increase in inhibition; each of the cyclodextrin-modified rutins ACD-rutin, BCD-rutin, GCD-rutin, and HPGCD-rutin inhibited SARS-COV-2 replication by 40-50% over untreated.

Tables 6-7 provide clinical results of cyclodextrin-modified complexes of rutin produced using various production methods (Table 6) and using various cyclodextrins (Table 7) in a SARS-COV-2 in vitro model and a diabetes mouse model. Note that the sample codes of Table 6 relate to the codes from Table 4, while the sample codes of Table 7 relate to the codes of Table 5. As can be seen in Tables 6 and 7, all forms of cyclodextrin-conjugated rutin had higher activity than unconjugated rutin in an in vitro model of SARS-COV-2. Also, among the cyclodextrin conjugated forms of rutin, the highest activity in the in vitro model of SARS-COV-2 was obtained using betacyclodextrin conjugated to rutin. There was variability in activity among the betacyclodextrin conjugates produced under various reaction conditions used for conjugation. The activity was obtained in a reaction that was mixed for 6 hours at a 1:1 molar ratio, in a solvent of 1:1 Water 95% EtOH, at 40° C., under atmospheric pressure that was oven dried subsequent to mixing. Similarly, rutin cyclodextrin conjugates had higher activity than unconjugated rutin in a streptozotocin diabetes mouse model.

TABLE 6

| | | | | |
|---|---|---|---|---|
| Sample | Final Concentration | Percent SARS- | Dose of Gavage | Difference in 4 h |
| code from | SARS-CoV-2 in vitro | CoV-2 in vitro | Diabetes Model | Blood Glucose |
| Table 4 | Model μg/ml | Inhibition Assay | mg/kg | vs. Untreated* |

β-Cyclodextrin-Rutin Complexes Produced Using Various Molar Ratios

| | | |
|---|---|---|
| 1 | 25 | 76.76 |
| 2 | 25 | 103.29 |
| 3 | 25 | 95.88 |

β-Cyclodextrin-Rutin Complexes Produced Using Prior Art “Kneading” Method

| | | |
|---|---|---|
| 4 | 1 | 42.6 |

β-Cyclodextrin-Rutin Complexes Produced Using Various Solvents, Molar Ratios, and Reaction Times

| | | | | |
|---|---|---|---|---|
| 5 | | | 25 | 109.34 |
| 6 | | | 25 | 99.39 |
| 7 | 1 | 41.09 | 25 | 101.42 |
| 8 | 1 | 38.5 | | |
| 9 | 1 | 49.16 | | |
| 10 | 1 | 42.99 | | |
| 11 | 1 | 31.42 | | |
| 12 | 1 | 27.5 | | |
| 13 | 1 | 34.09 | | |
| 14 | 1 | 28.83 | | |
| 15 | 1 | 32.42 | | |
| 16 | 1 | 27.83 | | |

β-Cyclodextrin-Rutin Complexes Produced Using Various Reaction Conditions

| | | |
|---|---|---|
| 17 | 1 | 40.42 |
| 18 | 1 | 38.42 |
| 19 | 1 | 30.16 |
| 20 | 1 | 30.09 |
| 21 | 1 | 43.42 |
| 22 | 1 | 40.42 |
| 23 | 1 | 37.09 |
| 24 | 1 | 33.42 |
| 25 | 1 | 46.09 |
| 26 | 1 | 43.01 |
| 27 | | |
| 28 | | |
| 29 | 1 | 50.09 |
| 30 | 1 | 44.09 |
| 31 | 1 | 36.46 |
| 32 | 1 | 35.49 |
| 33 | 1 | 30.09 |
| 34 | 1 | 31.09 |
| 35 | 1 | 27.06 |
| 36 | 1 | 30.16 |
| 37 | 1 | 35.75 |
| 38 | 1 | 34.09 |
| 39 | 1 | 30.75 |
| 40 | 1 | 28.09 |
| 41 | | |
| 42 | | |
| 43 | | |
| 44 | | |
| 45 | | |
| 46 | | |
| 47 | | |
| 48 | | |
| 49 | | |
| 50 | | |

β-Cyclodextrin-Rutin Complexes Produced Using Higher Temperature and Pressure

51
52
53
54

β-Cyclodextrin-Rutin Complexes Produced Using Higher Temperature at
Various Reactant Concentrations and Reaction Times

| | | |
|---|---|---|
| 55 | | |
| 56 | 1 | 43.75 |

Unconjugated Rutin

| | | | | |
|---|---|---|---|---|
| 57 | 1 | 23.29 | 25 | 89 |

TABLE 7

| | | | | |
|---|---|---|---|---|
| Sample | Final Concentration | Percent SARS- | Dose of Gavage | Difference in 4 h |
| code from | SARS-CoV-2 in vitro | CoV-2 Inhibition, | Diabetes Model | Blood Glucose |
| Table 5 | Model μg/ml | in vitro Model | mg/kg | vs. Untreated* |

γ-Cyclodextrin-Rutin Complexes

| | | |
|---|---|---|
| 1 | 1 | 37.6 |
| 2 | 1 | 31.93 |

Hydroxypropyl-γ-Cyclodextrin-Rutin Complexes

| | | |
|---|---|---|
| 3 | 1 | 46.69 |
| 4 | 1 | 40.36 |

α-Cyclodextrin-Rutin Complexes

| | | |
|---|---|---|
| 5 | 1 | 41.26 |

HPBCD-Rutin Complexes

| | | | | |
|---|---|---|---|---|
| 6 | 1 | 45.45 | 25 | 104.95 |
| 7 | 1 | 41.78 | 25 | 124.28 |
| 8 | 1 | | 25 | 117.44 |

HEBCD-Rutin Complex

| | |
|---|---|
| 9 | 1 |

SBBCD-Rutin Complex

| | |
|---|---|
| 10 | 1 |

Unconjugated Rutin

| | | | | |
|---|---|---|---|---|
| 11 | 1 | 23.29 | 25 | 89 |

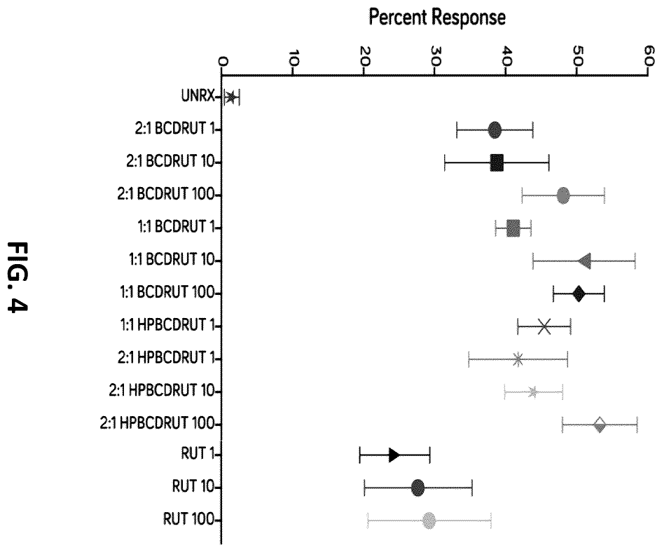

In FIG. 4, additional cyclodextrin-modified rutins were analyzed an in vitro model of SARS-COV-2 described previously. In addition, cyclodextrin-modified rutins in which the molar ratio of the cyclodextrin to rutin was varied were also analyzed. As can be seen, unmodified rutin as well as all cyclodextrin-modified rutins tested inhibited SARS-COV-2 replication in vitro at all concentrations tested. In addition, a synergistic effect was observed for all cyclodextrin-modified rutins over unmodified rutin.

### Example 6

Eczema is a common chronic relapsing disorder. Primary lesions at presentation include erythematous macules, papules, and vesicles. Infection and/or excoriation result in secondary lesions that include lichenification and significant intercellular edema of the epidermis. Atopic dermatitis, the most common form of eczema and chronic inflammatory skin diseases, is characterized by a disturbance of epidermal-barrier function resulting in intensely pruritic and chronic eczematous plaques. Current AD therapy is reactive, where flares are treated through symptomatic management with emollients and topical corticosteroids. However, these medications have limited effects and, in the case of corticosteroids, are associated with a rebound effect. Moreover, there are long-term side-effects of corticosteroids, including skin atrophy, infection susceptibility, and adrenal suppression, which underscore the need for reducing life-time corticosteroid burden, especially in pediatric patients.

In this Example, anti-PHR agents of the present disclosure were screened in an oxazolone-induced murine model of chronic atopic dermatitis. Mice were sensitized with a single topical application of 5% oxazolone to the flank, followed by challenges with 0.2% topical oxazolone every other day for total of 4 weeks. An ethanol-treated vehicle group served as the unaffected control. Induced mice developed chronic AD, as evidenced by persistent pruritic chronic dermatosis and increased epidermal hyperplasia, development of parakeratotic scales, and lymphocytic infiltrates. A Clinical Activity Score, equal to the sum of the severity of 5 signs and symptoms (itch, erythema/hemorrhage, edema, excoriation/erosion and scaling/dryness), each of which is graded on a 3-point scale (0-none, 1-mild, 2-moderate, 3-severe), was used to evaluate therapeutic efficacy of the anti-PHR agents.

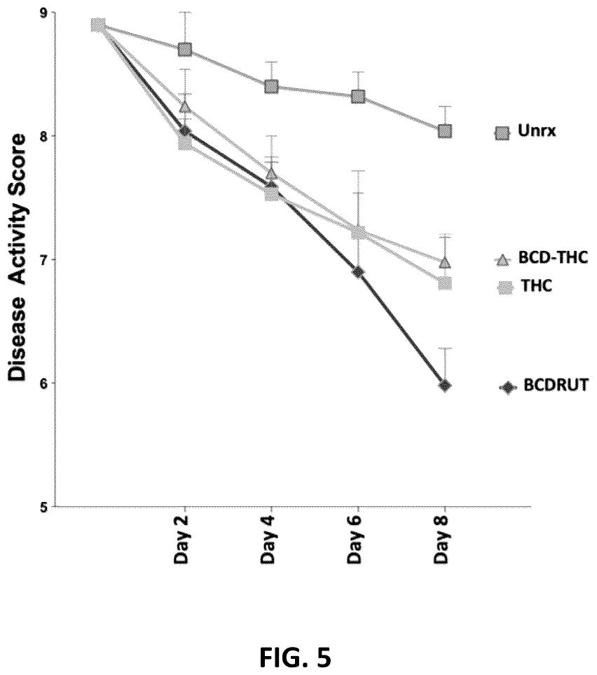

FIG. 5 demonstrates that mean Clinical Activity Scores were decreased by 33.9% for the topically applied anti-PHR agent (BCD Rutin). This level of efficacy was significantly higher than other emollient therapies tested, including (but not limited to) Aveeno (data not shown). The anti-PHR agents decreased histological and biochemical aspects of atopic dermatitis in this model. Skin biopsies of anti-PHR-treated mice had significant decreases in hyperplasia and dermal inflammatory infiltrates. No abnormal findings were observed in the morphological and histological investigations of all tissues from AD mice that were treated with anti-PHR.

### Example 7

Table 8 contains a summary of survival data obtained for various anti-PHR agents alone, various anti-viral agents alone, as well as various combinatorial therapies containing at least one anti-PHR agent and at least one anti-viral agent, in the in vivo COVID-19 mouse model described previously. The anti-PHR agents used are in native or cyclodextrin-modified forms. A formulation containing a combination of various anti-PHR agents was also analyzed; this combination (labeled as “APHR Combination 1”) included Betacyclodextrin Curcumin, Quercetin, Hesperidin, Betulinic acid, Zinc acetate, Baicalin, Tannic acid, Ebselen, Bisabolol, green tea extract, Betacyclodextrin-Tetrahydrocurcumin, Betacyclodextrin-Catechin, Glycyrrhetinic Acid, rose extract, licorice extract, Salvia miltiorrhiza extract, and Houttuynia cordata Thunb. Extract (HCT).

TABLE 8
Effects of APHR Agents on Survival in Covid-19 Mouse Model

| | Dose | | Survival |
|---|---|---|---|
| Agent | (mg/kg) | Days Treated | (%) |
| Hydroxypropylbetacyclodextrin Curcumin | 200 | Daily, Pretreatment to Day 7 | 100 |
| Hydroxypropylbetacyclodextrin Curcumin | 66 | Daily, Pretreatment to Day 7 | 100 |
| Quercitin | 200 | Daily, Pretreatment to Day 7 | 100 |
| Quercitin | 66 | Daily, Pretreatment to Day 7 | 80 |
| Hydroxypropylbetacyclodextrin Curcumin + Quercitin | 100 of each | Daily, Pretreatment to Day 7 | 100 |
| APHR Combination 1 | 200 | Daily, Pretreatment to Day 7 | 100 |
| APHR Combination 1 | 50 | Daily, Pretreatment to Day 7 | 80 |
| Betacyclodextrin Rutin | 200 | Daily, Day 1 post-treatment to 7 | 100 |
| Betacyclodextrin Rutin | 50 | Daily, Day 1 post-treatment to 7 | 100 |
| Betacyclodextrin Rutin | 25 | Daily, Day 1 post-treatment to 7 | 80 |
| Betacyclodextrin Rutin + Green tea extract | 25 of each | Daily, Day 1 post-treatment to 7 | 80 |
| Betacyclodextrin Rutin + betacyclodextrin Curcumin | 25 of each | Daily, Day 1 post-treatment to 7 | 100 |
| Betacyclodextrin Rutin + betacyclodextrin Quercitin | 25 of each | Daily, Day 1 post-treatment to 7 | 100 |
| Betacyclodextrin Rutin + Glycyrrhetinic acid | 25 of each | Daily, Day 1 post-treatment to 7 | 100 |
| Hydroxypropylbetacyclodextrin Rutin | 50 | Daily, Day 1 post-treatment to 7 | 100 |
| Hydroxypropylbetacyclodextrin Rutin + Hydroxypropylbetacyclodextrin Curcumin | 25 of each | Daily, Day 1 post-treatment to 7 | 100 |
| Hydroxypropylbetacyclodextrin Rutin + Remdesivir | 25 of each | Daily, Day 1 post-treatment to 7 | 100 |
| Betacyclodextrin Rutin + Hesperidin | 25 of each | Daily, Day 1 post-treatment to 7 | 80 |
| Betacyclodextrin Rutin | 12.5 | Daily, Day 2 post-treatment to 7 | 80 |
| Betacyclodextrin Rutin + Baicalin | 12.5 of each | Daily, Day 2 post-treatment to 7 | 100 |
| Betacyclodextrin Rutin + Rose extract | 12.5 of each | Daily, Day 2 post-treatment to 7 | 100 |
| Betacyclodextrin Rutin + betacyclodextrin Curcumin | 20 and 5 | Daily, Day 2 post-treatment to 7 | 100 |
| Betacyclodextrin Rutin + Atorvastatin | 12.5 of each | Daily, Day 2 post-treatment to 7 | 100 |
| Betacyclodextrin Rutin + Licorice extract | 12.5 of each | Daily, Day 2 post-treatment to 7 | 83 |
| Betacyclodextrin Rutin + Betacyclodextrin Licorice extract | 12.5 of each | Daily, Day 2 post-treatment to 7 | 83 |
| Betacyclodextrin Rutin + Nafamostat mesylate | 12.5 of each | Daily, Day 2 post-treatment to 7 | 83 |
| Remdesivir (positive control) | 200 | Daily, Pretreatment to Day 7 | 100 |
| Remdesivir (positive control) | 50 | Daily, Day 1 post-treatment to 7 | 100 |
| Untreated (negative control) | | | 40-60 |
| | | | (mean = 50) |

As can be seen, a wide variety of anti-PHR agents have been tested in the in vivo COVID-19 mouse model and shown to be effective in preventing morbidity and mortality from COVID-19, both as single agents and in combinations of two or more agents.

While the above disclosures describe the inventive concept(s) in conjunction with the specific experimentation, results, and language set forth herein, it is evident that many alternatives, modifications, and variations will be apparent to those skilled in the art. Accordingly, it is intended to embrace all such alternatives, modifications, and variations that fall within the spirit and broad scope of the present disclosure.

1. A method of producing at least one cyclodextrin-modified agent, the method comprising the steps of:
(a) combining at least one agent with a cyclodextrin (CD) to form a mixture;
(b) incubating the mixture at a temperature above room temperature and at a pressure above atmospheric pressure for a sufficient amount of time to form a cyclodextrin-modified agent.

2. The method of claim 1, wherein the cyclodextrin-modified agent has enhanced bioavailability when compared to the agent alone.

3. The method of claim 1, wherein at least one of:
the cyclodextrin is selected from the group consisting of alpha-cyclodextrin (ACD), beta-cyclodextrin (BCD), hydroxyethyl beta-cyclodextrin (HEBCD), hydroxypropyl beta-cyclodextrin (HPBCD), sulfobutyl beta-cyclodextrin (SBCD), gamma-cyclodextrin (GCD), or hydroxypropyl gamma-cyclodextrin (HPGD); and/or
the at least one agent is selected from the group consisting of rutin, sodium rutin, curcumin, tetrahydrocurcumin, quercetin, hesperidin, baicalin, green tea extract, rose extract, betulinic acid, tannic acid, bisabolol, licorice extract, a modified form thereof, and combinations thereof.

4. The method of claim 3, wherein the at least one agent is selected from the group consisting of rutin, curcumin, tetrahydrocurcumin, and combinations thereof.

5. The method of claim 3, wherein the agent comprises quercetin and curcumin.

6. The method of claim 3, wherein the at least one cyclodextrin-modified agent is selected from the group consisting of ACD-curcumin, BCD-curcumin, HEBCD-curcumin, HPBCD-curcumin, SBCD-curcumin, GCD-curcumin, HPGD-curcumin, or BCD-tetrahydrocurcumin.

7. The method of claim 3, wherein the at least one cyclodextrin-modified agent is selected from the group consisting of ACD-quercetin, BCD-quercetin, HEBCD-quercetin, HPBCD-quercetin, SBCD-quercetin, GCD-quercetin, or HPGD-quercetin.

8. The method of claim 3, wherein the at least one cyclodextrin-modified compound is selected from the group consisting of ACD-rutin, BCD-rutin, HEBCD-rutin, HPBCD-rutin, SBCD-rutin, GCD-rutin, or HPGD-rutin.

9. The method of claim 1, wherein in step (b):
the temperature is in a range of from about 20° C. to about 300° C.;
the pressure is in a range of from about 15 PSI to about 200 PSI; and
the amount of time is in a range of from about 0.1 hour to about 18 hours.

10. The method of claim 9, wherein in step (b), the amount of time is in a range of from about 0.1 hours to about 1 hour.

11. The method of claim 1, wherein in step (a), the agent and cyclodextrin are combined in a cyclodextrin: agent molar ratio of from about 1:1 to about 3:1.

12. The method of claim 1, wherein step (a) is further defined as combining at least one agent with a cyclodextrin (CD) and a solvent to form a mixture.

13. The method of claim 12, wherein the solvent is selected from the group consisting of ethanol, water, methanol, acetone, and combinations thereof.

14. A pharmaceutical composition, comprising:
a composition comprising at least one cyclodextrin-modified agent produced by the method of claim 1; and
a pharmaceutically acceptable carrier.

15. The pharmaceutical composition of claim 14, wherein the pharmaceutical composition is formulated for sublingual, oral, parenteral, and/or topical administration.

16. A method, comprising:
administering the pharmaceutical composition of claim 14 to a patient in need thereof.

17. The method of claim 16, wherein the pharmaceutical composition is administered sublingually.

18. The method of claim 17, further defined as a method of treating or reducing the occurrence of a viral infection in a patient.

19. (canceled)

20. The method of claim 16, further defined as a method of treating or reducing the occurrence of diabetes in a patient.

21. The method of claim 16, wherein the method is further defined as a method of treating or reducing the occurrence of at least one skin condition, and wherein the pharmaceutical composition is administered topically.

22-43. (canceled)
